(19) United States
(12) Patent Application Publication
(10) Pub. No.: US 2025/0064851 A1
(43) Pub. Date: Feb. 27, 2025

(54) Methods of Producing Plasma or Serum and Uses Thereof

(71) Applicant: THE REGENTS OF THE UNIVERSITY OF CALIFORNIA, Oakland, CA (US)

(72) Inventor: Justin Karlin, Los Angeles, CA (US)

(21) Appl. No.: 18/913,003

(22) Filed: Oct. 11, 2024

Related U.S. Application Data
(63) Continuation of application No. PCT/US23/65701, filed on Apr. 13, 2023.
(60) Provisional application No. 63/330,584, filed on Apr. 13, 2022.

(51) Int. Cl. A61K 35/16; A61F 9/00; A61K 9/00; A61K 47/10; A61K 47/36

(52) U.S. Cl. CPC A61K 35/16; A61F 9/0008; A61K 9/0048; A61K 47/10; A61K 47/36

(57) ABSTRACT

A method of producing serum or plasma comprises the steps of drawing a blood sample from a subject; adding erythrocyte sedimentation-accelerating agent(s) and an anticoagulant to the blood sample; placing the mixture in a vessel with an inclined well and allowing the phase separation of the solution into an erythrocyte phase and a plasma or serum phase, producing an erythrocyte-depleted serum or plasma; and collecting the erythrocyte-depleted serum or plasma. A method of producing an ophthalmic solution (“eye drops”) meant for lubricating the eye of a subject comprises the steps of drawing a blood sample from a subject; adding erythrocyte sedimentation-accelerating agent(s) and an anticoagulant to the blood sample; placing the mixture in a vessel with an inclined well and allowing the phase separation of the solution into an erythrocyte phase and a plasma or serum phase, producing an erythrocyte-depleted serum or plasma; collecting the erythrocyte-depleted serum or plasma; treating the plasma with a platelet activating substance or agent; and applying the erythrocyte-depleted serum or plasma to the eye of the subject. A kit provides components, diagrams, compositions and instructions for performing the methods.

### CROSS-REFERENCE TO REPLATED APPLICATIONS

This application is a Continuation-in-Part (CIP) claiming priority to International Application PCT/US2023/065701, filed Apr. 13, 2023, which claims priority to U.S. Provisional Application No. 63/330,584, filed Apr. 13, 2022, each of which are incorporated herein by reference in their entireties.

### BACKGROUND OF THE INVENTION

With respect to blood products, serum may be described as the aqueous component of blood that is free of erythrocytes (red blood cells), leukocytes (white blood cells), thrombocytes (platelets) and clotting factors. Plasma is the aqueous component of blood that is free of erythrocytes. Platelet-rich plasma (PRP) is plasma that is processed to increase the concentration of platelets. PRP typically contains at least twice the concentration of platelets of whole blood. Plasma rich in growth factors (PRGF) is plasma or PRP where the platelets have released their contents, including growth factors.

Ocular surface disease affects hundreds of millions of people worldwide. Autologous blood derived eye drops (ABED), including serum eye drops (ASED) and plasma eye drops (PRP or PRGF), are topical ophthalmic drops that are made from a patient's own blood. ASED, PRP and PRGF, when applied to the eye surface, have been demonstrated to benefit patients with a wide variety of ocular surface diseases, including common conditions such as dry eye syndrome, as well as more rare entities such as non-healing corneal epithelial defect, neurotrophic keratopathy, and aniridia associated keratopathy.

ABED are typically prepared in specialty laboratories by highly trained individuals. Sterile conditions are critical in the preparation of ABED, as the contents of ABED (such as proteins, glucose, lipids and salts) make for a highly favorable environment for the growth of microorganisms such as bacteria and fungi. To ensure sterile conditions, the preparation of ABED is usually carried out by savvy personnel who adhere to strict protocols. These protocols may include the following steps: donning of personal protective equipment by the preparer, surface decontamination, use of sterilized laboratory equipment and preparation in special spaces, such as under a laminar flow hood (so as to protect open containers from contamination by airborne spores, bacteria and other microorganisms during the preparation process). The laboratory personnel involved in ABED preparation often require extensive training in order to ensure adherence to stringent sterility guidelines. The law in some countries and in some American states, requires that those who compound blood into eye drops obtain licensing by specialty organizations.

In general, the steps in the process of laboratory preparation of ABED are as follows. Peripheral venous blood is collected by phlebotomy into vacuum tubes, with or without anticoagulant. Anticoagulated (PRP and PRGF) or non-anticoagulated (ASED) whole blood is then centrifuged one (ASED and plasma) or more (PRP and PRGF) times. In other words, centrifugation is performed to remove erythrocytes and leukocytes, and may be performed in order to concentrate platelets (i.e., thrombocytes). Under sterile conditions, the supernatant is isolated, often by decanting or pipetting, and then the resulting liquid may be filtered through a sieve to remove leukocytes, microorganisms and/or residual red blood cells. The filtered supernatant may be treated with exogenous substances (such as calcium chloride) or with physical manipulation (for instance, shear forces, heating or cooling) to stimulate the release of growth factors from thrombocytes (as in the case of PRGF). The resulting product may then be diluted and then this solution is distributed into one or more eye dropper bottle(s). This product, in turn, dispensed to the patient.

In spite of being a highly effective ophthalmic therapeutic modality, ABED are expensive and not widely available. Laboratory preparation requires specialty laboratory expertise, equipment, and, in some places, licensing. All of these factors drive up production costs for those involved in ABED production (ex. mobile phlebotomists, compounding pharmacies and laboratories). This cost is passed on to patients, who may pay hundreds to thousands of dollars for a three-month supply. Patients often pay this price as an out-of-pocket expense, as the vast majority of third-party payers do not cover ABED. High prices lead to lower demand and thus a lower supply. This relative dearth of producers means that ABED producers are few and far between. And even if a producer is identified, it may take days to weeks from the time a patient's eligibility is determined to the time this patient actually receives these eye drops.

There remains a need in the art for rapid and less expensive methods to produce ABED, such as ASED, PRP, and PRGF. These methods must be as sterile as or more sterile than current methods. The present invention addresses this unmet need.

Beyond ophthalmology, platelet-rich plasma (PRP) and plasma rich in growth factors (PRGF) have found widespread applications in various medical fields, including orthopedics, sports medicine, dermatology, and wound care. In orthopedics and sports medicine, PRP has been utilized to treat a range of musculoskeletal conditions, such as tendinopathies, ligament injuries, and osteoarthritis. The growth factors and bioactive proteins present in PRP are believed to promote tissue healing, reduce inflammation, and accelerate recovery times. Similarly, in dermatology, PRP has gained popularity for its potential in hair restoration treatments and skin rejuvenation procedures, leveraging its ability to stimulate collagen production and tissue regeneration.

Wound care is another area where PRP and PRGF have shown promise. These autologous blood products have been applied to chronic wounds, such as diabetic foot ulcers and pressure sores, with the aim of enhancing healing rates and reducing the risk of complications. The concentrated growth factors in PRP and PRGF are thought to stimulate angiogenesis, promote cell proliferation, and modulate the inflammatory response, all of which are crucial for effective wound healing.

Despite the growing interest and potential benefits of PRP and PRGF in these diverse medical fields, the challenges of production and availability persist, mirroring those seen in ophthalmology. The need for specialized equipment, trained personnel, and sterile laboratory conditions makes the preparation of these autologous products time-consuming, expensive, and often inaccessible to many patients and healthcare providers. This limitation is particularly pronounced in settings where immediate treatment could be beneficial, such as sports injuries or acute wound care scenarios.

The high cost of PRP and PRGF treatments in these fields is often borne entirely by patients, as many insurance providers consider these therapies experimental or investigational. This financial burden can limit access to potentially beneficial treatments, particularly for chronic conditions that may require multiple applications over time. Furthermore, the time delay between blood draw and product preparation can be a significant drawback in acute care settings, where rapid intervention could potentially improve outcomes.

As in ophthalmology, there is a pressing need across these medical specialties for methods to produce PRP and PRGF more quickly, efficiently, cost-effectively, and safely. Innovations that could simplify the preparation process, reduce the need for specialized equipment and personnel, and maintain or improve sterility standards would have far-reaching implications. Such advancements could potentially democratize access to these autologous therapies, allowing for their more widespread use in various clinical settings, from large medical centers to small clinics and even point-of-care applications.

The development of rapid, cost-effective, and sterile methods for producing PRP and PRGF would not only benefit patients and healthcare providers but could also facilitate more robust clinical research. With easier access to standardized preparations, researchers could more readily conduct large-scale studies to further elucidate the efficacy of these treatments across different medical conditions. This, in turn, could lead to refined treatment protocols, potentially broader insurance coverage, and ultimately, improved patient care across multiple medical disciplines.

### SUMMARY OF THE INVENTION

In one aspect, the present invention relates to a method of producing autologous blood derived eye drops (ABED), including serum or plasma (i.e., PRP and PRGF) eye drops. The method comprises the steps of drawing a blood sample from a subject in to a hermetically sealed and sterile sedimentation vessel; mixing in this vessel of the collected blood sample with a sedimentation-accelerating agent, with or without an anticoagulant; with the optional step of adding a preservative to prevent microbial growth; incubation of this mixture in the sedimentation vessel where the wall (i.e. the vessel's interior surface) is inclined with respect to the force of gravity; phase separation of this mixture via sedimentation under the force of gravity into (1) clarified supernatant (i.e. serum or plasma) and (2) settled erythrocyte phases; collection of the supernatant, i.e. the aqueous component of blood that has been depleted of erythrocytes; optional in-line filtration of this supernatant to remove residual erythrocytes, leukocytes and/or microbes; optional dilution of this supernatant; optional activation of platelets retained within a plasma supernatant; optional analysis of the plasma supernatant for quality control, examination for contamination; aliquoting of the plasma or serum into one or more eye dropper bottles; venting of accumulated air pressure from the closed system and/or closed device (e.g., device 600); detaching the eye dropper bottle from the apparatus; attaching an additional eye dropper bottle to the apparatus for additional collection and aliquoting; labeling of the eye dropper bottles with a patient-specific label; and direct dispensing of the eye dropper bottle(s) to a patient.

In some embodiments, the method further comprises the step of passing the supernatant through at least one filter that removes any contaminating microorganisms or any residual erythrocyte aggregates and leukocytes. In some embodiments, the sedimentation-accelerating agent is immobilized on a solid-phase immobilization material. In some embodiments, the sedimentation-accelerating agent is immobilized on a solid-phase immobilization material that is coupled to the interior of the vessel wall. In some embodiments, the preservative is immobilized on a solid phase immobilization material. In some embodiments, the preservative is immobilized on a solid phase immobilization material that is affixed to the interior of the vessel wall. In some embodiments, the preservative is immobilized on a solid phase immobilization material that is affixed to the interior of the eye dropper bottle's wall.

In another aspect, the present invention relates to a device that is used for producing ABED, including plasma [including PRP or PRGF] or serum eye drops. Key aspects of the device are, first, that prior to and after the collection of blood, the device remains a hermetically sealed, closed system and/or closed device (e.g., device 600). In other words, the patient's blood that enters the system and/or device (e.g., device 600) does not contact the ambient environment during the serum or plasma preparation process. The second key aspect of this method and device is that separation of whole blood into an erythrocyte phase and a serum or plasma phase is carried out using the process of erythrocyte sedimentation. The third key aspect of this method and device is that the process of erythrocyte sedimentation is accelerated by mixing the blood with a sedimentation-accelerating agent: a polymer, protein, enzyme, bioactive lipid, and/or small molecule sedimentation-accelerating agent. The fourth key aspect of this method and device is that sedimentation is further accelerated by incubating the blood and sedimentation-accelerating agent mixture in a vessel that is held at an incline with respect to force of gravity. Phase separation of blood by sedimentation (into erythrocyte and plasma/serum layers) is known in the art to accelerate when the interior wall of the vessel containing the sedimenting blood is inclined with respect to the force of gravity, a phenomenon called the ‘Boycott effect.’

In some embodiments, the sedimentation-accelerating agent used in the method and device comprises a nanoparticle, polymer, macromolecule, protein, proteolytic enzyme, bioactive lipid, a small molecule or some combination thereof. In some embodiments, the sedimentation-accelerating agent comprises a polymer selected from the group consisting of glycerin, polyethylene glycol (PEG), polypropylene glycol, polysorbate, gelatin, hydroxymethylcellulose, hydroxyethylcellulose, hydroxypropyl methylcellulose, polyvinyl alcohol, dextran, hyaluronic acid, hydroxyethyl starch, povidone (polyvinylpyrrolidone, PVP), poly-l-glutamic acid, poly(ethylene oxide)-poly(propylene oxide)-poly(ethylene oxide) triblock copolymer, and tetrameric (poly)albumin. In some embodiments, the sedimentation-accelerating agent comprises a protein selected from the group consisting of fibrinogen, immunoglobulin G, immunoglobulin M, C-reactive protein, haptoglobin, α₁-acid glycoprotein, and albumin. In some embodiments, the sedimentation-accelerating agent comprises a proteolytic enzyme selected from the group consisting of chymotrypsin, trypsin, bromelain, and neuraminidase. In some embodiments, the sedimentation-accelerating agent comprises one or more small molecules selected from the group consisting of phosphatidic acid and lysophosphatidic acid. In some embodiments, the sedimentation-accelerating agent comprises a bioactive lipid selected from the group consisting of phosphatidylcholine and lysophosphatidylcholine. In some embodiments, the sedimentation-accelerating agent comprises dextran having a molecular weight ranging from 40 kilodaltons (kDa) to 1000 kDa. In some embodiments, the sedimentation-accelerating agent comprises one or more compounds that fall into one or more of the following groups: polymer, macromolecule, protein, enzyme, proteolytic enzyme, bioactive lipid, and small molecule.

In some embodiments, the anticoagulant that is mixed with blood is a sodium citrate solution. In some embodiments, the anticoagulant is ethylenediaminetetraacetic acid (EDTA). In some embodiments the anticoagulant is heparin solution.

In some embodiments, the preservative mixed with the blood product is benzalkonium. In some embodiments, the preservative is sodium perborate. In some embodiments the preservative is polyquaternium-1. In some embodiments the preservative is sodium chlorite. In some embodiments, no preservative is included.

In some embodiments, the sedimentation vessel is a cylinder. In some embodiments, the sedimentation vessel is a syringe. In some embodiments, during the sedimentation process, the axis of the sedimentation vessel is held at an angle that is between 0 degrees and 90 degrees to the force of gravity. In some embodiments, the interior wall of the blood collection vessel is maintained at such an inclined position using a stand or a rack on which the vessel rests. In some embodiments, the vessel has an inclined interior wall, as in a cone. In some embodiments, during the process of sedimentation, this cone-shaped vessel's vertex is pointed toward the earth's surface and the cone's axis is oriented parallel to the force of gravity. In some embodiments, the incline of the vessel's interior wall is modified during the erythrocyte sedimentation process by reorienting the vessel.

In another aspect, the present invention relates to a method of creating a lubricant and therapeutic agent for the eye of a subject, the method comprising the steps of: drawing a blood sample from a subject; adding an agent to the blood sample to accelerate sedimentation of the erythrocytes, thereby causing phase separation of the blood into erythrocyte and an erythrocyte-depleted serum or plasma; collecting the erythrocyte-depleted serum or plasma in an eye dropper bottle; and applying the erythrocyte-depleted serum or plasma to the eye surface of the subject. In some embodiments, the vessels are hermetically sealed and the step of collecting the erythrocyte-depleted serum or plasma comprises the step of transferring the erythrocyte-depleted serum or plasma into the eye dropper bottle that is used to administer these drops directly to the patient's eye surface.

In another aspect, the present invention relates to a kit for producing autologous blood derived eye drops from a patient's blood sample. In some embodiments, the kit comprises a sedimentation vessel, a collection vessel, an erythrocyte sedimentation-accelerating agent (as described elsewhere herein), anticoagulant solution (as described elsewhere herein) and preservative solution (as described elsewhere herein). In some embodiments, the sedimentation vessel is a blood sedimentation vessel, and the collection vessel is a plasma/serum collection vessel. In some embodiments, the sedimentation vessel and/or collection vessel may or may not be pre-filled with a sedimentation-accelerating agent in solution, with or without anticoagulant solution, with or without a preservative solution. In some embodiments, the kit further comprises one or more selected from the group consisting of: a three-way stopcock; a venipuncture set; one or more syringes; one or more needles; one or more filters (for removal from the plasma or serum any residual leukocytes or residual erythrocytes or both); one or more racks or stands for keeping the sedimentation vessel at a specific incline in relation to the force of gravity during the sedimentation process; one or more one-way valves; a plasma/serum collection vessel which is also a removable eye dropper bottle element; a coupler connecting the eye dropper bottle element to the three-way stopcock or to the filter; an eye-dropper bottle cap; labels for ensuring the chain of custody; and instructions for use of the kit. In some embodiments, the components of the kit are sterilized.

The elements within the aforementioned kit may be assembled into a device. In some embodiments, this device is used to prepare and dispense sterile autologous blood derived eye drops that are intended to be used to lubricate and treat the eye surface of a patient.

Although the method and device as described and the elements within the aforementioned kit that comprise the method and device are suited for the preparation of autologous plasma or serum eye drops, the resulting plasma or serum that is produced by this device may be used for other purposes known in the art. For instance, in some embodiments, this device can be used to prepare serum or plasma for analyses known in the art such as measurement of electrolyte levels, glucose levels, protein levels, or biomarker levels. In some embodiments, this device can be used in settings where analysis is needed but no centrifuge or laboratory preparation is available. In some embodiments this device can be used to prepare platelet rich plasma that can be applied topically to the skin or can be used for dermatologic treatments wherein platelet rich plasma is used to treat the dermis. In some embodiments, the device can be used to prepare platelet rich plasma or other derivatives of platelet rich plasma that can be used for joint injections (e.g., hip, knee, or shoulder) in the context of sports medicine, pain management, rheumatology or orthopedic surgery. In some embodiments, the platelet rich plasma resulting from the use of device can be processed and used as a hemostatic agent, for instance as platelet-rich fibrin. In some embodiments, the resulting platelet-rich fibrin can be used as a topical agent in wound care, to promote wound healing. In some embodiments, platelet-rich plasma or derivative preparations can be used during surgery as a hemostatic agent or as a tissue glue. In some embodiments, the platelet-rich plasma or derivative preparations can be used for intradermal, subdermal or other soft tissue injection. In some embodiments, the platelet rich plasma or derivative preparations can be used as an intradermal injection to stimulate the growth of hair follicles.

### BRIEF DESCRIPTION OF THE DRAWINGS

The following detailed description of preferred embodiments of the invention will be better understood when read in conjunction with the appended drawings. For the purpose of illustrating the invention, embodiments are shown in the drawings which are presently preferred. It should be understood, however, that the invention is not limited to the precise arrangements and instrumentalities of the embodiments shown in the drawings.

FIG. 1 depicts a flowchart of a representative method of producing erythrocyte-depleted plasma or serum.

FIG. 2 depicts a flowchart of a representative method of producing and utilizing erythrocyte-depleted plasma or serum.

FIG. 3 depicts a drawing of an exploded view of a representative device of the invention.

FIG. 4 depicts a representative drawing of a device of the present invention assembled and connected to a tube for drawing blood.

FIG. 5 depicts a representative drawing of a device of the present invention in the process of drawing blood into a plunger, where the blood mixes with the sedimentation-accelerating agent and optional anticoagulant and/or preservatives.

FIG. 6 depicts a representative drawing of a device of the present invention being rotated so that the erythrocytes sediment with the vessel at an inclined angle in relation to the direction of the force of gravity.

FIG. 7 depicts a representative drawing of a device of the present invention being up-righted after sedimentation, rotation of the stopcock to fluidly connect the syringe and bottle, and expulsion of the plasma from the syringe style sedimentation vessel into the eye dropper bottle style collection vessel.

FIG. 8 depicts a representative drawing of the eye dropper bottle being removed after the plasma has been introduced into the bottle.

FIG. 9 depicts representative sedimentation rates upon addition of polymer solutions to citrate anticoagulated blood. Erythrocyte sedimentation rate (mm/hr) was determined by the Westergren method. Data are presented as mean with standard deviation; n=3. Abbreviations: DEX70=dextran 70 kDa; DEX150=dextran 150 kDa; DEX500=dextran 500 kDa; PEG10=polyethylene glycol 10; PEG35=polyethylene glycol 35.

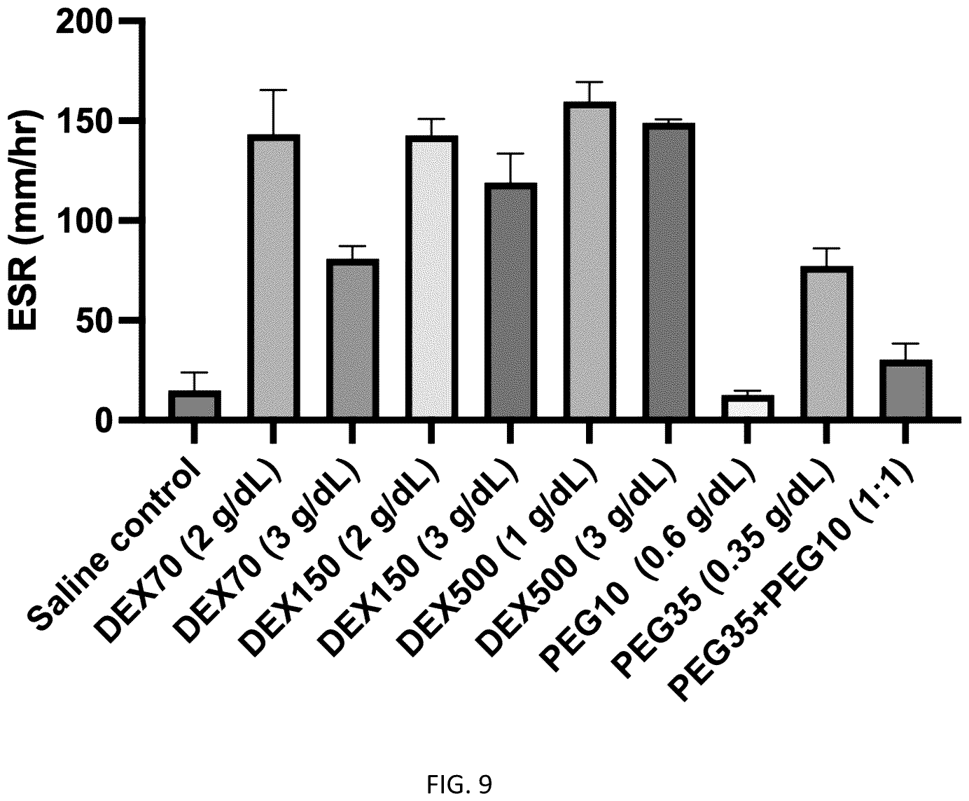

FIG. 10 depicts representative platelet counts of various plasma samples, demonstrating that sedimentation of blood leads to increased platelet concentration (in k/μL) in supernatant plasma. Platelet concentrations of plasma from blood sedimented in the presence of various polymers are also shown. Data are presented as mean with standard deviation. Abbreviations: DEX70=dextran 70 kDa; DEX150=dextran 150 kDa; DEX500=dextran 500 kDa; PEG10=polyethylene glycol 10; PEG35=polyethylene glycol 35.

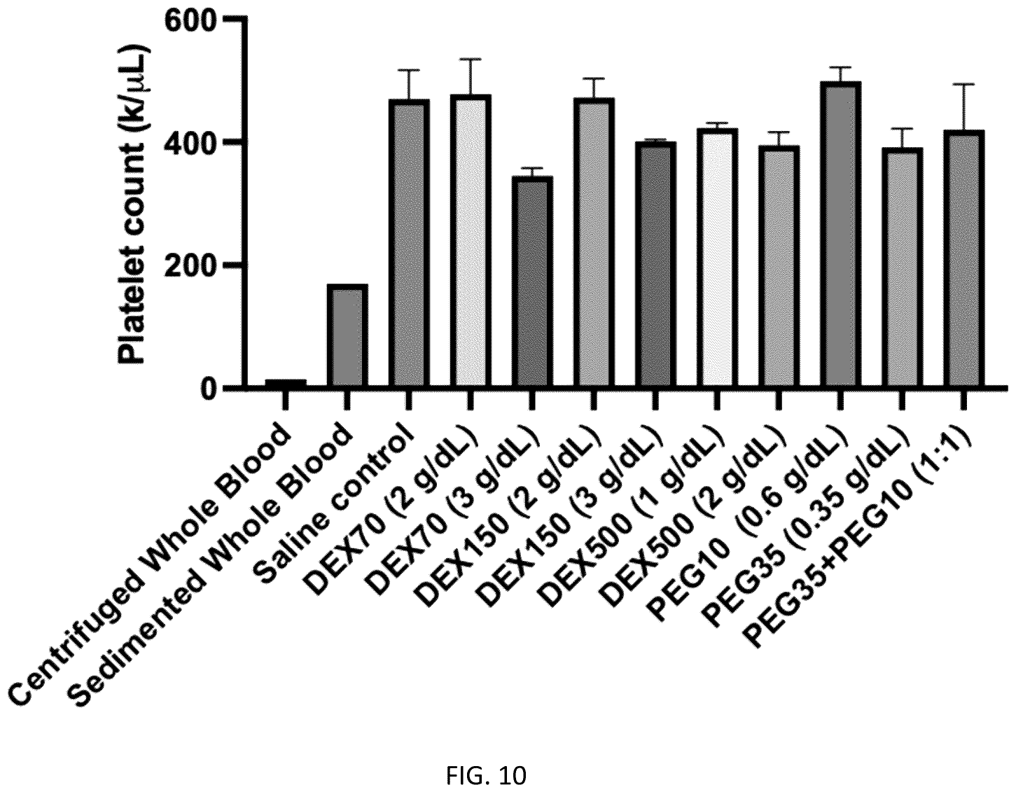

FIG. 11 depicts representative results demonstrating the impact of the Boycott effect. Whole anticoagulated blood was allowed to sediment for 30 minutes in vessels held at various angles (x-axis labels) in relation to the force of gravity. Yield (y-axis) is the volume of plasma recovered as a proportion of initial whole human blood. The yield of whole anticoagulated blood is compared to that of whole anticoagulated blood mixed with the polymer dextran 500 kDa (1.5 g/dL). Yield was greater at 30 degrees than it was at 90 degrees (i.e., upright vessel). Compared to whole anticoagulated blood, yield was greater for blood mixed with polymer.

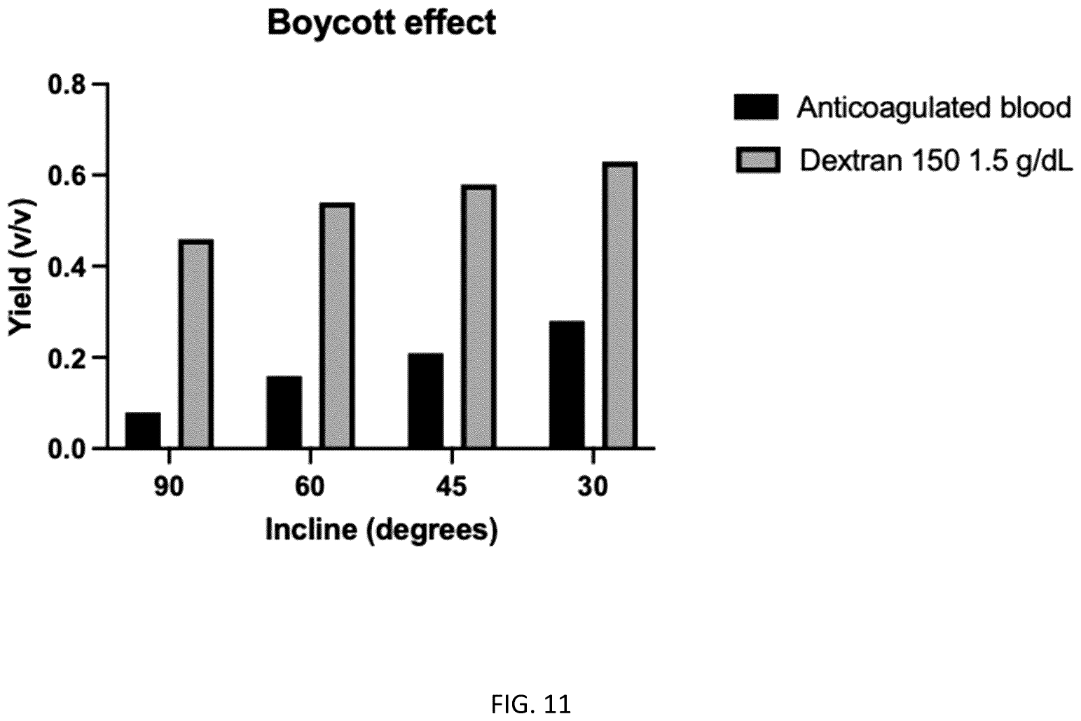

FIG. 12 depicts representative results demonstrating the impact of the Boycott effect. Whole anticoagulated blood was allowed sediment for 30 minutes in vessels held at various angles (x-axis labels) in relation to the force of gravity. Yield (y-axis) is the volume of plasma recovered as a proportion of initial whole human blood. The yield of whole anticoagulated blood is compared to that of whole anticoagulated blood mixed with the polymer dextran 500 kDa (1 g/dL). Yield was greater at 30 degrees than it was at 90 degrees (i.e., upright vessel). Compared to whole anticoagulated blood, yield was greater for blood mixed with polymer.

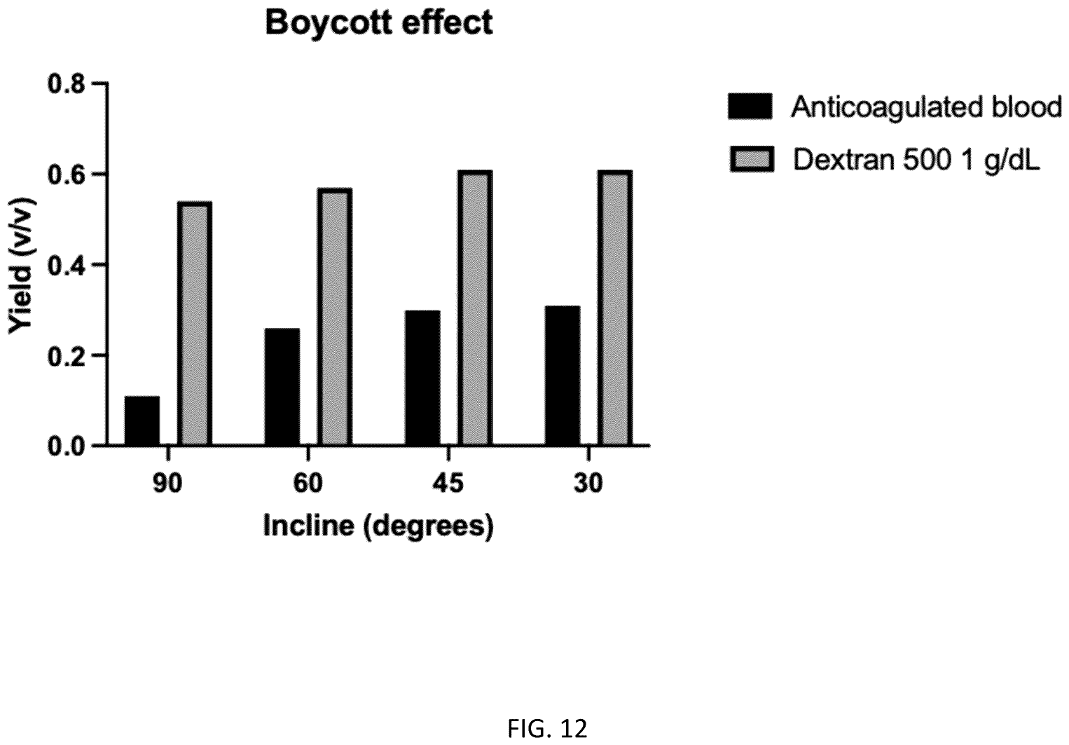

### DETAILED DESCRIPTION

It is to be understood that the figures and descriptions of the present invention have been simplified to illustrate elements that are relevant for a clear understanding of the present invention, while eliminating, for the purpose of clarity, many other elements found in routine laboratory preparation of blood products such as serum, plasma and cellular fractions. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not explicitly provided herein. The disclosure herein is directed to all such variations and modifications to such elements and methods known to those skilled in the art.

As used herein, each of the following terms has the meaning associated with it in this section. Unless defined otherwise, all technical and scientific terms used herein generally have the same meaning as commonly understood by one of ordinary skills in the art to which this invention belongs.

The term “plasma” refers to the aqueous fraction of blood that is free of erythrocytes (red blood cells, RBC) and leukocytes (white blood cells, WBC). The term “serum” refers to the aqueous fraction of blood that is free of erythrocytes, leukocytes and coagulation factors, including platelets and protein factors involved in the coagulation cascade. Plasma therefore encompasses serum. For all topically applied ophthalmic purposes described herein, eye drop preparations that are made from plasma are expected to behave biochemically and pharmacologically identically to serum eye drops. The two terms, “plasma” and “serum,” may thus be used interchangeably herein.

The term erythrocyte aggregate may refer to red blood cells that are grouped in a pattern known as rouleaux, or erythrocytes that are grouped together in other patterns.

The term erythrocyte sedimentation may refer to the process by which red blood cells descend under the force of gravity, thereby phase separating blood into an erythrocyte fraction and a plasma or serum fraction.

The articles “a” and “an” are used herein to refer to one or to more than one (i.e., to at least one) of the grammatical object of the article. By way of example, “an element” means one element or more than one element.

As used herein, the term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5,from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based in part on the unexpected result that common, safe ingredients used widely in topical lubricant eye drop preparations, such as povidone or dextran, are potent aggregators and accelerators of sedimentation of red blood cells that, at certain concentrations and under certain conditions, can greatly accelerate erythrocyte sedimentation.

The present invention is based in part also on the unexpected result that an erythrocyte sedimentation vessel that has an internal surface that is maintained at an incline with respect to the force of gravity during the sedimentation process will lead to an accelerated rate of erythrocyte sedimentation (when compared to sedimentation in non-inclined vessels).

The present invention is based in part also on the unexpected result that preparation of plasma by erythrocyte sedimentation by the method described herein leads to an increased platelet concentration compared to preparation of plasma by centrifugation.

The present invention is based in part also on the unexpected result that the method disclosed herein produces autologous blood derived eye drops (both serum and plasma eye drops and variants thereof, such as PRP and PRGF) faster, less expensively and with greater efficiency than most current methods.

### Methods of Producing Serum or Plasma

In some aspects, the present invention relates to a method of producing an erythrocyte-depleted, filtered serum and/or erythrocyte-depleted, filtered plasma. In some embodiments, the invention relates to a method of producing a topical serum or plasma ophthalmic solution.

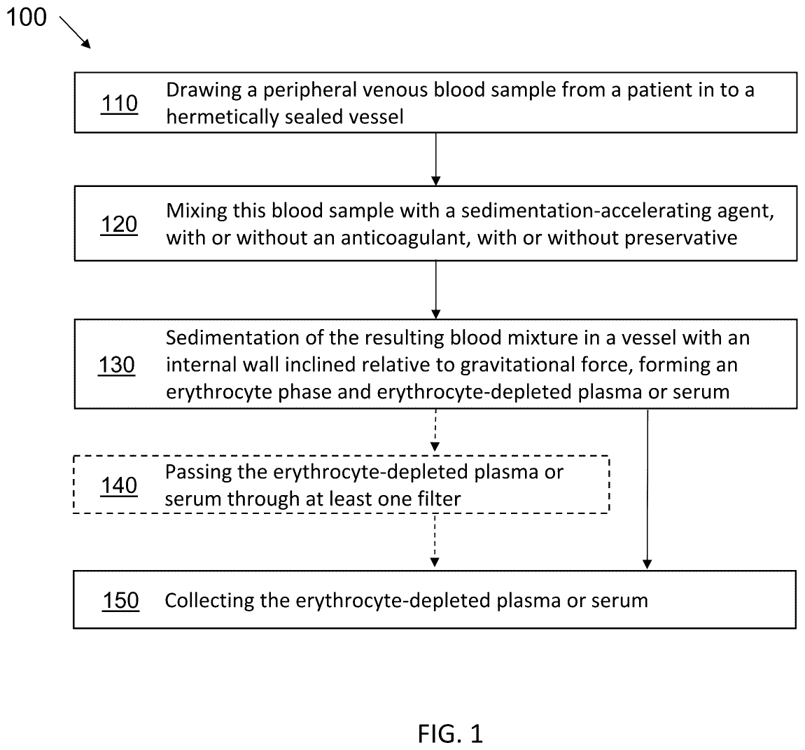

Exemplary method 100 is provided in FIG. 1. In step 110, a venous blood sample is drawn from a subject into a hermetically sealed vessel. In step 120, a sedimentation-accelerating agent, with or without an anticoagulant, with or without preservative is mixed with the blood sample to form an erythrocyte aggregate and an erythrocyte-depleted plasma. In step 130, the solution is allowed to sediment with the vessel wall at an incline in relation to the force of gravity, and the erythrocyte aggregates will descend and the erythrocyte-depleted plasma or serum will ascend. In optional step 140, the erythrocyte-depleted plasma is passed through at least one filter wherein leukocytes or residual erythrocytes are removed. In step 150, the erythrocyte-depleted plasma is collected. In some embodiments, the erythrocyte-depleted plasma or serum is an ophthalmic solution, and In some embodiments the solution is collected in an eye dropper bottle. In various embodiments, the entire method is performed without exposure of the blood, sedimentation-accelerating agent, and plasma or serum to the ambient atmosphere.

In some embodiments of step 110, a blood sample may be collected in any routine manner. In some embodiments, the blood sample is drawn from a vein of an individual (such as the external jugular vein, antecubital veins, existing central lines, or the wrist area, to name a few). In another embodiment, a blood sample may be collected from a bag of stored, previously collected anticoagulated whole blood, for instance citrated blood previously collected for donation. In some embodiments the blood is freshly collected. In another embodiment, the blood is previously collected for donation and has been refrigerated.

The volume of blood withdrawn from the subject is limited by what is safe, feasible and tolerable for each individual patient. In some embodiments, the ideal blood volume to be collected from the subject is more than 10 mL but less than 500 mL. In some embodiments, the minimum blood volume of 10 mL allows for a sufficient amount of plasma or serum to be collected to make a clinically relevant amount of serum eye drops. In some embodiments, the volume of blood withdrawn is collected in and fills a 10 mL syringe. In some embodiments, the volume of blood withdrawn is collected in and fills a 25 ml syringe. In some embodiments, the volume of blood withdrawn is collected in and fills a 50 mL syringe.

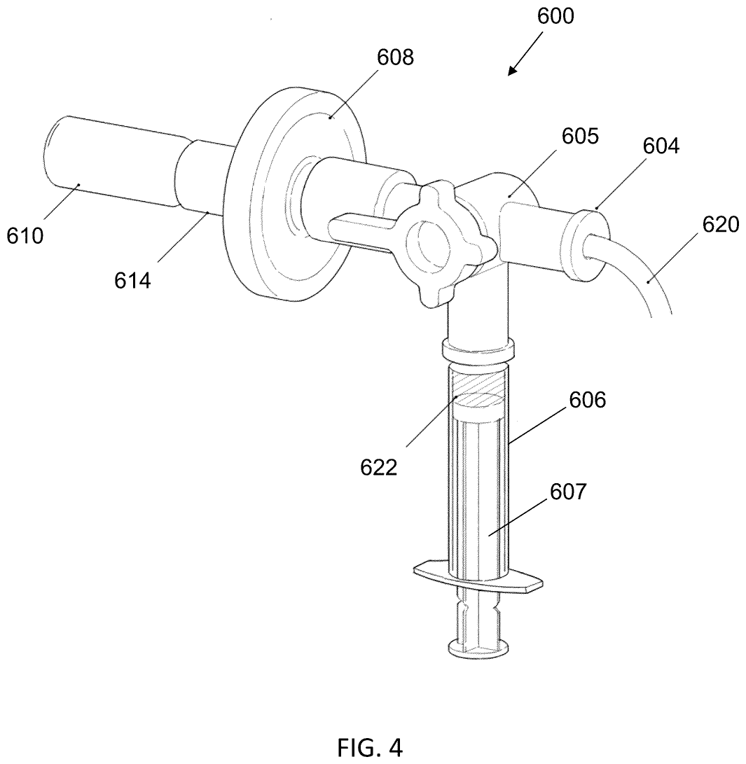

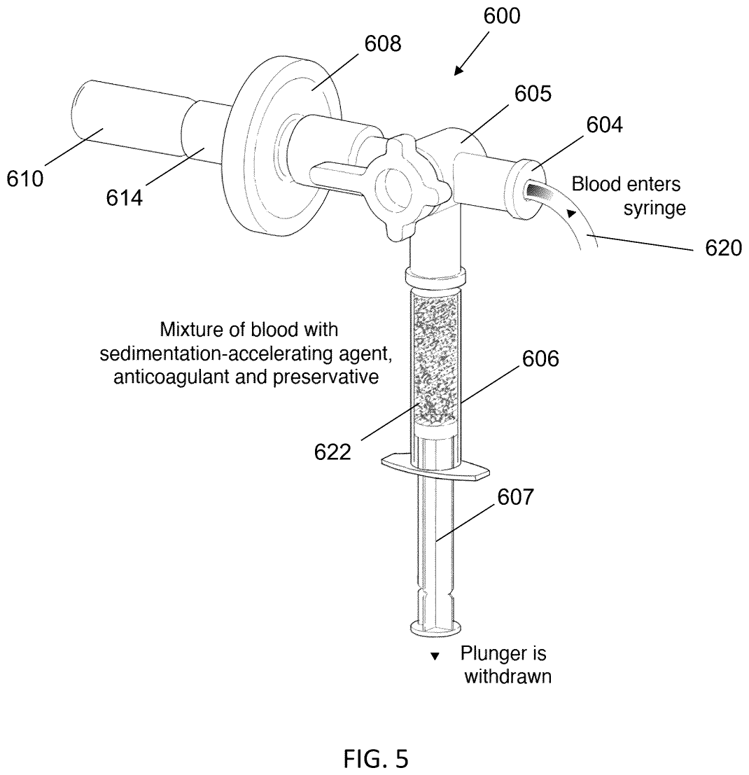

In some embodiments, the blood is drawn using a standard butterfly needle venipuncture set and the blood is collected initially in a Luer lock syringe. In some embodiments, the blood is collected into a hermetically-sealed, sterile system and/or device, such as device 600 of the present invention. In some embodiments, the blood never comes in contact with the ambient environment. In some embodiments, the blood is collected directly into a collection vessel where erythrocyte sedimentation will occur. In another embodiment, the blood is drawn into a blood donation collection bag. In another embodiment, the blood is drawn into a Luer lock syringe (See for example FIG. 4) which is connected via Luer lock to a three-way stopcock. In some embodiments, the blood passes through the three-way stopcock to enter the syringe. In some embodiments, the blood flows through a one-way valve which is connected to the three-way stopcock into the Luer lock syringe, when the plunger of the syringe is withdrawn (See for example FIG. 5).

In some embodiments, step 110 further comprises the step of examining the blood for the presence of pathogens, toxins or poisons. The term ‘pathogens’ within the meaning of the present invention includes infectious or other biological disease-causing or-promoting agents that may have adverse effects in humans and, especially when eye drops are used, could jeopardize the health of the ocular surface (i.e., cornea or conjunctiva) or the health of the patient or the success of the therapy. In other words, step 110 comprises the step of examining patient blood for pathogenic agents which could possibly lead to the formation of eye diseases (ex. keratitis, conjunctivitis, endophthalmitis), organ damage (ex. endocarditis) or systemic diseases (ex. sepsis). Examples of pathogens, toxins or poisons in this setting include inorganic or organic compounds, heavy metals, small molecule poisons, toxic enzymes and protein toxins, bacterial lipopolysaccharide, bacteria, fungi, viruses, prions, and parasites. In some embodiments, examination for pathogens, toxins or poisons may also be performed before the blood is withdrawn from the subject. In some embodiments, a sub-sample of the blood is removed from the blood sample without exposing the sample to the ambient environment.

In step 120, a sedimentation-accelerating agent is mixed with blood, and the reaction between the two increases the rate at which erythrocytes separate from surrounding blood plasma or serum. In some embodiments, the aforementioned sedimentation-accelerating agent induces aggregation via agglutination of erythrocytes. In some embodiments, the aforementioned sedimentation-accelerating agent accelerates the formation of erythrocyte aggregates, including stereotypical rouleaux, and thereby increases the erythrocyte sedimentation rate (ESR). In various embodiments, the sedimentation-accelerating agent is sterile and mixed with the blood without exposure to the ambient environment.

In some embodiments of step 120, the sedimentation-accelerating agent is added to the erythrocyte sedimentation vessel before the blood draw. In some embodiments, the sedimentation-accelerating agent is present in the erythrocyte sedimentation vessel at the time of the blood draw (See for example FIG. 4). In some embodiments, the blood enters the syringe and is immediately contacted with the sedimentation-accelerating agent (See for example FIG. 5). In some embodiments, the blood is drawn into a syringe first, then the sedimentation-accelerating agent is added to the blood within the syringe and the two are mixed. In some embodiments, this sedimentation-accelerating agent is added to the blood-containing syringe via a three-way stopcock.

In some embodiments, the sedimentation-accelerating agent in step 120 comprises a polymer, macromolecule, protein, proteolytic enzyme, bioactive lipid, or small molecule capable of inducing sedimentation of red blood cells in the blood sample. In some embodiments, the sedimentation-accelerating agent increases the sedimentation rate of erythrocytes by increasing erythrocyte aggregation. In some embodiments, the sedimentation-accelerating agent comprises a mixture of sedimentation-accelerating agents. In some embodiments, the sedimentation-accelerating agent comprises a single sedimentation-accelerating agent. In some embodiments, the sedimentation-accelerating agent comprises one, two, or three or more than three different sedimentation-accelerating agents.

In some embodiments, the sedimentation-accelerating agent comprises a polymer or macromolecule. Exemplary polymers include, but are not limited to, glycerin, polyethylene glycol (PEG), polypropylene glycol, polysorbate, gelatin, hydroxymethylcellulose, hydroxyethylcellulose, hydroxypropyl methylcellulose, polyvinyl alcohol, dextran, hyaluronic acid, starches such as hydroxyethyl starch (Hetastarch), povidone (polyvinylpyrrolidone, PVP), poly-1-glutamic acid, poloxamers (amphiphilic block copolymers consisting of poly(ethylene oxide)-poly(propylene oxide)-poly(ethylene oxide) triblock copolymer (PEO-PPO-PEO)), and polymers comprising multiple units of proteins, such as tetrameric (poly)albumin.

In some embodiments, the polymer is conjugated to a nanoparticle which allows for finer control and tuning of the sedimentation accelerating agent's chemical properties such as solubility, hydrodynamic radius, and net charge. In some embodiments, conjugation of the polymer allows for finer control of rheological properties such as viscosity.

In some embodiments, the sedimentation-accelerating agent comprises a polymer having a hydrodynamic radius of at least 4 nm. In some embodiments, the sedimentation-accelerating agent comprises a polymer having a radius of between 2 nm and 4 nm. In some embodiments, the sedimentation-accelerating agent comprises a polymer having a radius of about 3 nm.

In some embodiments, the sedimentation-accelerating agent comprises poly(ethylene glycol) (PEG) having a molecular weight between 5 kDa and 500 kDa. In some embodiments, the PEG has a molecular weight between 15 kDa and 90 kDa. In some embodiments, the PEG has a molecular weight between 20 kDa and 70 kDa. In some embodiments, the PEG has a molecular weight between 20 kDa and 60 kDa. In some embodiments, the PEG has a molecular weight between 25 kDa and 50 kDa. In some embodiments, the PEG has a molecular weight between 30 kDa and 40 kDa. In some embodiments, the PEG has a molecular weight of about 35 kDa. In some embodiments, there would be a mixture of PEG of different molecular weights. In some embodiments, the sedimentation-accelerating agent comprises a mixture of PEG 10 kDa and PEG 35 kDa.

In some embodiments, the sedimentation-accelerating agent is dextran. In some embodiments, the dextran has a molecular weight greater than 40 kDa. In some embodiments, the dextran has a molecular weight between 70 kDa and 500 kDa. In some embodiments, the dextran has a molecular weight between 150 kDa and 500 kDa. In some embodiments, the dextran has a molecular weight greater than 1000 kDa. In some embodiments, the dextran has a molecular weight of exactly 70 kDa or 150 kDa or 500 kDa. In some embodiments, the dextran has a molecular weight that is between 60 kDa and 80 kDa, or approximately between 100 kDa and 200 kDa, or between 450 kDa and 650 kDa. In some embodiments, there would be a mixture of dextran of different molecular weights. In some embodiments, the sedimentation-accelerating agent comprises a mixture of dextran 150 kDa and 500 kDa. In some embodiments, the sedimentation-accelerating agent comprises a mixture of dextran 70 kDa, 150 kDa and 500 kDa. In some embodiments, the dextran has a molecular weight between 500 kDa and 1000 kDa.

In some embodiments, the sedimentation-accelerating agent comprises a protein. Exemplary proteins include, but are not limited to fibrinogen, globulins such as immunoglobulin G (IgG) or immunoglobulin M (IgM), C-reactive protein, haptoglobin, α₁-acid glycoprotein, and albumin.

In some embodiments, the sedimentation-accelerating agent comprises a proteolytic enzyme. Exemplary suitable proteolytic enzymes include, but are not limited to, chymotrypsin, trypsin, bromelain, and neuraminidase.

In some embodiments, the sedimentation-accelerating agent comprises a small molecule. Exemplary small molecules include, but are not limited to, phosphatidic acid and lysophosphatidic acid.

In some embodiments, the sedimentation-accelerating agent comprises a bioactive lipid. Exemplary bioactive lipids include, but are not limited to, phosphatidylcholine and lysophosphatidylcholine.

In some embodiments, the sedimentation-accelerating agent comprises dextran (of any of the aforementioned molecular weights described elsewhere herein) and the concentration of dextran is between 0.01 mg/dL and 10 g/dL. In some embodiments, the sedimentation-accelerating agent comprises dextran and the concentration of dextran is between 0.1 g/dL and 5 g/dL. In some embodiments, the sedimentation-accelerating agent comprises dextran and the concentration of dextran is between 0.5 g/dL and 3 g/dL. In some embodiments, the sedimentation-accelerating agent comprises dextran and the concentration of dextran is 1 g/dL or 2 g/dL.

In some embodiments, the sedimentation-accelerating agent comprises PEG and the concentration of the sedimentation-accelerating agent is between 0.01 g/dL and 1 g/dL. In some embodiments, the sedimentation-accelerating agent comprises PEG and the concentration of the sedimentation-accelerating agent is between 0.2 g/dL and 0.7 g/dL. In some embodiments, the sedimentation-accelerating agent comprises PEG and the concentration of the sedimentation-accelerating agent is between 0.25 g/dL and 0.5 g/dL. In some embodiments, the sedimentation-accelerating agent comprises PEG and the concentration of the sedimentation-accelerating agent is between 0.3 g/dL and 0.4 g/dL. In some embodiments, the sedimentation-accelerating agent comprises PEG and the concentration of the sedimentation-accelerating agent is about 0.35 g/dL.

In some embodiments, the sedimentation-accelerating agent comprises the enzyme bromelain and the concentration of bromelain therein is between 0.01 g/dL (0.1 mg/mL) and 1 g/dL (10 mg/mL). In some embodiments, the concentration of bromelain is between 0.2 g/dL and 0.8 g/dL. In some embodiments, the concentration of bromelain is between 0.3 g/dL and 0.7 g/dL.

In some embodiments, the sedimentation-accelerating agent comprises the enzyme neuraminidase at a concentration between 1 U/mL and 100 U/mL. In some embodiments, the concentration of neuraminidase therein is between 10 and 80 U/mL. In some embodiments, the neuraminidase concentration is between 20 and 60 U/mL. In some embodiments, the neuraminidase concentration is between 30 and 40 U/mL. In some embodiments, the neuraminidase concentration is about 33 U/mL.

In some embodiments, the sedimentation-accelerating solution comprises a mixture of two or more different types of sedimentation-accelerating agents. In some embodiments, the sedimentation-accelerating agents used are a combination of a polymer and a protein in solution. In some embodiments the sedimentation-accelerating agent is a conjugate of a polymer covalently linked to a protein. In some embodiments the sedimentation-accelerating agent comprises a conjugate of a polymer covalently linked to a nanoparticle. In another embodiment, the sedimentation-accelerating agents used in the solution are a combination of a polymer and an enzyme. In some embodiments, there is a mixture of a polymer and a small molecule. In some embodiments, there is a mixture of a polymer and a bioactive lipid. In some embodiments, the sedimentation-accelerating agents included in the solution are a mixture of a polymer, a protein and a small molecule. In some embodiments, there is a mixture of a polymer, a small molecule and an enzyme. In some embodiments, the sedimentation-accelerating agents are a mixture of a polymer, a protein and an enzyme. In some embodiments, the sedimentation-accelerating agents contained in the solution are a mixture of a polymer, a protein, a small molecule and an enzyme. In some embodiments, the sedimentation-accelerating agents included in the sedimentation accelerating solution is a permutation of mixtures of the following: nanoparticle, polymer, protein, macromolecule, small molecule, enzyme, bioactive lipid.

In some embodiments, the sedimentation-accelerating agent is used in a free form, e.g., in aqueous solution. In some embodiments, the sedimentation-accelerating agent is conjugated to solid-phase immobilization materials (such as polymer beads, magnetic beads, plastic beads, silica or alumina beads, cellulose, nanoparticles, etc.). In some embodiments, a portion of the sedimentation-accelerating agent is present as a free agent and another portion is conjugated to beads to facilitate aggregation. The beads may be magnetic beads and, in such a case, a magnetic field can be applied during incubation of the blood sample with the sedimentation-accelerating agent to further accelerate aggregation.

In some embodiments, an electromagnetic field may be used to accelerate aggregation and sedimentation of the aggregated RBC. In some embodiments, an enzyme such as neuraminidase is used to remove sialic acid residues from the RBC surface, thus increasing the electrostatic charge of the RBC surface; an electromagnetic field may then be used to segregate red blood cells from the surrounding plasma or serum.

In some embodiments, in vitro alteration of the pH of the blood sample may be used to accelerate aggregation and sedimentation of erythrocytes. In some embodiments the pH of the blood sample is made more alkaline by the addition of hydroxyl ions, and this in turn is done to accelerate erythrocyte sedimentation. In some embodiments, the pH of the blood sample is made more alkaline with the addition of an organic base and in some embodiments the pH of the blood sample is made more alkaline by the addition of an inorganic base. In some embodiments, once sedimentation is deemed to be complete, the pH of the resulting plasma is brought back to neutral (i.e., pH 7) with the addition of a neutralizing acid.

In some embodiments, step 120 further comprises the step of adding an anticoagulant to the blood sample. In some embodiments, the anticoagulant prevents platelet removal from the erythrocyte-depleted plasma, allowing platelets to remain in the plasma solution. In some embodiments, the anticoagulant prevents coagulation of the blood sample. In some embodiments, the anticoagulant comprises sodium citrate. In some embodiments, heparin is the anticoagulant used. In another embodiment, ethylenediaminetetraacetic acid (EDTA) is the anticoagulant used. In some embodiments, the anticoagulant is present in solution with the sedimentation-accelerating agent. In some embodiments, the anticoagulant is added to the blood sample before the sedimentation process is initiated.

In some embodiments, step 120 further comprises the step of mixing the sedimentation-accelerating agent and the blood sample. In some embodiments, the solution is mixed by inverting the syringe multiple times (e.g., between 2 and 30 times). In some embodiments, the solution is mixed by shaking or vibrating the syringe. In some embodiments, the addition of a larger volume of blood to a smaller volume of aggregating solution will accomplish mixing without needing an additional mixing step. In some embodiments, the blood and sedimentation-accelerating solutions are mixed by passing the blood and sedimentation accelerating agent mixture back and forth multiple times between two syringes via a Luer connector, such as a three-way stopcock. In some embodiments, the aforementioned mixing steps also include an anticoagulant.

Step 130 comprises the step of allowing the erythrocytes to sediment. In some embodiments, the mixture of blood and sedimentation-accelerating agent (with or without anticoagulant, with or without preservative) is incubated below room temperature (−20 to 20° C.), at room temperature (20 to 25° C.) or higher that room temperature (such as from 25-50° C.) for a suitable period of time to induce erythrocyte sedimentation. For example, the sample can be allowed to stand for up to 120 minutes (such as from 1 to 120 minutes and all values therebetween). However, it is preferable to allow the samples to stand for less than 60 minutes. For example, the samples can stand for 55, 45, 30, 25, 20, 15, 10 or 5 minutes. In some embodiments, the sample is allowed to stand for 10-30 minutes and all values and ranges therebetween. If the sedimentation-accelerating agent is conjugated to a magnetic solid phase immobilization material, a magnetic field may be applied to further reduce the time of preparation. If the sedimentation-accelerating agent causes an increase in the electrostatic charge on the surface of the RBC, an electromagnetic field may be used to accelerate RBC aggregation, plasma/serum separation, and thus will reduce the time of preparation.

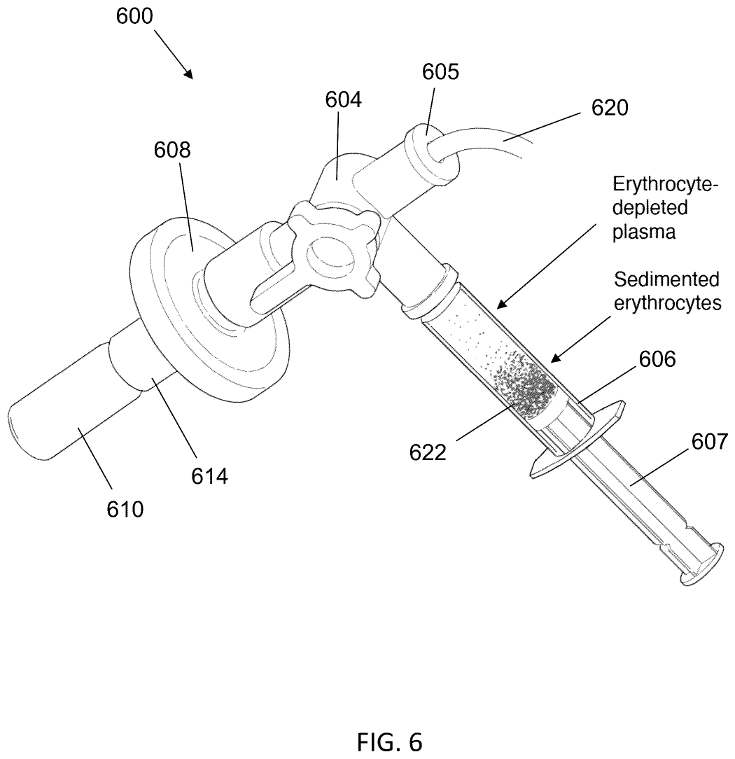

In some embodiments, step 130 further comprises the step of keeping the interior walls of the sedimentation vessel at an incline with respect to gravity (FIG. 6). This step leverages a phenomenon known as the Boycott effect, whereby tilting a cylindrical vessel containing a heterogeneous sedimenting solution will result in an increased rate of sedimentation. In some embodiments, step 130 comprises the step of keeping a cylindrical erythrocyte sedimentation vessel's long axis at an incline with respect to the force of gravity. In some embodiments, the orientation of such a sedimentation vessel's long axis is at an angle of between 0 and 90 degrees with respect to the direction of gravity. In some embodiments, the sedimentation vessel is cone shaped and, during sedimentation, the vertex of the cone is pointed toward the center of the earth and the axis of the cone is oriented parallel to the force of gravity. In some embodiments, the angle of the sedimentation vessel is altered during the sedimentation process. In some embodiments, the angle of the sedimentation vessel is kept constant during the sedimentation process.

In some embodiments, the sedimentation occurs under the force of earth's gravity. In some embodiments, the blood sample is collected in a syringe which is oriented such that the erythrocyte aggregate settles generally on the plunger side of the syringe, and the erythrocyte-depleted plasma collects on the opposite side of the syringe. In some embodiments of the invention, the resulting clarified supernatant following sedimentation is centrifuged to separate, isolate or concentrate various other blood cell components. In some embodiments, it may be desirable to avoid additional preparation steps such as centrifugation. Avoiding such steps might be expected to decrease the time and equipment required to produce a point-of-care serum or plasma eye drop. Accordingly, in some embodiments, no extra-system steps, such as centrifugation, are performed. Accordingly, in some embodiments, the method can be used in settings where analysis or treatment is needed but no centrifuge or laboratory preparation is available.

There is no particular limit to the filter employed in optional step 140. In some embodiments, the filter is a commercially available syringe filter known to those of skill in the art. The typical filter pore size ranges from 0.1 microns to 50 microns. The typical syringe filter pore size used for filtering plasma or serum is approximately 5 microns. In some embodiments, a single filter is used. In another embodiment, multiple filters are used sequentially. In some embodiments, the multiple filters are connected to one another. In another embodiment, the multiple filters are connected to different parts of the apparatus, including to the sedimentation vessel, or to an inlet or outlet of an attached three-way stopcock, or to a one-way valve.

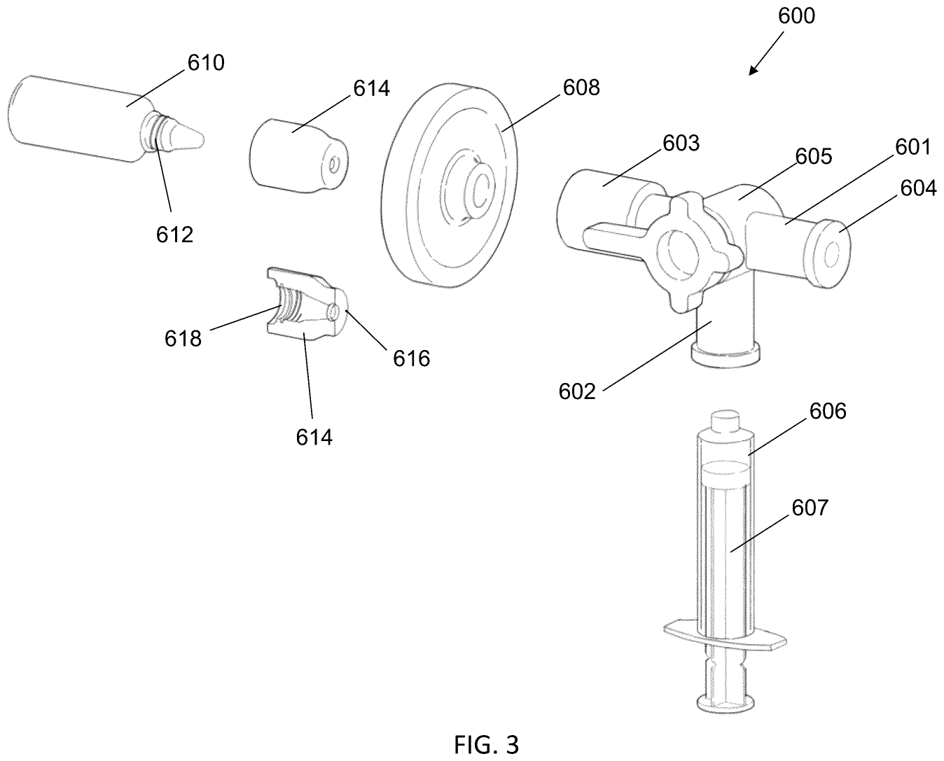

In some embodiments, step 150 comprises the step of passing the erythrocyte-depleted plasma or serum from the sedimentation vessel into a collection vessel. In some embodiments, the sedimentation vessel is connected to the sample collection vessel via a three-way stopcock (See for example FIG. 4). In some embodiments, there is a filter between the sedimentation vessel and the collection vessel. In some embodiments, the three-way stopcock's three ports connect (1) the incoming blood from a patient's vein, (2) a sedimentation vessel, and (3) a collection vessel (See for example FIG. 3). In some embodiments there is an in-line filter or multiple in-line filters between the sedimentation vessel and the collection vessel. In some embodiments, there is a specialized coupler that connects the three-way stopcock to the collection vessel. In some embodiments, this coupler connects the in-line filter to an eye dropper bottle style plasma collection vessel, as illustrated in FIGS. 3 and 4. In some embodiments, there is a one-way valve that allows fluid movement to proceed in only one direction, from the sedimentation vessel to the collection vessel.

In some embodiments of step 150, the flow of an erythrocyte-depleted plasma or serum from the sedimentation vessel into the collection vessel via a one-way valve results in an accumulation of pressure in the collection vessel. A key part of step 150 is that this accumulated pressure is vented by releasing air. In some embodiments, there is a three-way stopcock between the one-way valve and the collection vessel, and turning of the three-way stopcock knob can be used to vent air pressure in the collection vessel out of the third port of the three-way stopcock. In some embodiments, venting of aforementioned accumulated air in the collection vessel takes place when the collection vessel is sufficiently disengaged from the assembly. In some embodiments, venting of aforementioned accumulated air is through a filter meant to absorb liquid but allow the passage of air, preventing splashing or spilling of any serum or plasma onto the preparer. In some embodiments, the venting occurs through a one-way valve on the third port of the three-way stopcock such that air is allowed to vent out but not recirculate into the collection vessel.

In some embodiments, the collection vessel may be fully or partially evacuated prior to filling with serum or plasma. In some embodiments, the collection vessel may be pre-evacuated to induce flow of erythrocyte-depleted plasma or serum from the sedimentation vessel into the collection vessel. In some embodiments, the pressure differential between the sedimentation vessel and the collection vessel drives fluid movement and thus increases the efficacy of plasma or serum filtration through fluidly connected filter(s). In some embodiments, the collection vessel is pre-evacuated (or de-pressurized) in order to avoid accumulated pressure and to obviate a venting step. In some embodiments, the collection vessel is de-pressurized by manual action of the user. In some embodiments, prior to collection of the serum/plasma solution in the collection vessel, depressurization of the collection vessel is accomplished when the user evacuates air out of the collecting vessel by drawing air into a syringe connected to the collection vessel via a three-way stopcock. In some embodiments, the sample collection container produces negative pressure on the erythrocyte-depleted plasma to force the erythrocyte-depleted plasma through a flow control element or device, and through a filter, to draw the serum/plasma into the collection vessel.

In some embodiments, entry of plasma or serum into the collection vessel further comprises a flow rate control section preceding or following a connected filter. This flow rate control section may be internal or external to the collection vessel, i.e., the flow rate control element or device may be located inside or outside the collection vessel. In some embodiments, the flow rate control is integral with the sedimentation vessel. In some embodiments, the collection vessel incorporates an input flow rate control element.

In some embodiments, the sedimentation vessel is connected to the collection vessel (e.g., an eye dropper bottle in FIG. 3) via flow limiting tubing. In some embodiments, the flow limiting tubing maintains low shear stress as the erythrocyte-depleted plasma or serum flows through the device. This flow limitation minimizes disruption of any residual aggregates passing from the sedimentation vessel to the collection vessel. In this case, when filtration also occurs in-line, this allows for more efficient filtration of residual erythrocyte aggregates. In some embodiments, the pressure differential is controlled to induce a low velocity of the blood components beginning at the initial stage of filtering to minimize shear force on the cells, or impelling damage from collision with glass fibers of the filter assembly or with cell lodged in a tangle of glass fiber, in a manner to avoid excess hemolysis.

In some embodiments, the pressure differential across the filter may be limited to below 30 mm Hg per second. In some embodiments, the pressure differential across the filter may be limited to below 20 mm Hg per second. In some embodiments, the flow rate through the filter may be between about 2 and about 10 mL per minute. In some embodiments, the flow rate may be between about 3 and about 6 mL per minute. In some embodiments, the device may be constructed to produce a volume of between about 1 and about 40 mL filtrate. In some embodiments, the device may be constructed to produce a volume of about 15 mL filtrate. The limiting control element or device may be a tubular element between about ½ inch and about 4 inches in length and may have an internal diameter between about 0.008 and about 0.013 inch.

In some embodiments, neither air nor gas is permitted to enter any part of the sample collection container until the sedimentation, (optional) filtration, and collection processes have been completed. In some embodiments, the collection vessel is brought to atmospheric pressure by letting air at atmospheric pressure enter through a port covered in an airborne microbe, spore, and dust retaining filter. In some embodiments, the collection vessel is brought to atmospheric pressure by letting a medical, food, or breathing grade gas enter through a port optionally covered in an airborne microbe, spore, and dust retaining filter. Examples of acceptable medical, food, or breathing grade gasses include, but are not limited to, air, nitrogen, oxygen, carbon dioxide, argon, and combinations thereof. In some embodiments, the collection vessel may be detached from the remaining apparatus without exposure to atmospheric conditions. In some embodiments, the collection vessel is a container which is equipped with the necessary apparatus to apply the erythrocyte-depleted serum or plasma to the eye of a subject.

In step 150, in some embodiments of the invention, the serum/plasma collection vessel is a standard eye dropper bottle (See for example FIG. 3), with or without a nozzle, either 2.5, 5, 10, 20 or 30 mL in size. In some embodiments, the eye dropper bottle (See for example FIG. 4) is connected to the remainder of the system and/or device (e.g., device 600) via a coupler that fits a Luer connector on one end and an eye dropper nozzle on the other end. In other words, the sedimentation vessel is connected to the collection vessel via a coupler a simple screw mechanism, where the male component is the standard dropper nozzle of an eye dropper bottle and the “female” on the sample collection container is machined to accommodate the male component in a hermetically sealed manner (See for example FIGS. 3-8). In some embodiments the simple screw mechanism is coupled also to the three-way stopcock using a rubber gasket and a Luer lock mechanism. In some embodiments, the eye dropper bottle itself harbors a nozzle that fits a male Luer lock. In another embodiment, the eye dropper harbors a nozzle that fits a female Luer lock. In some embodiments, the eye dropper collection vessel is coupled directly to a filter which is sequentially connected to a three-way stopcock. In some embodiments, the eye dropper bottle collection vessel is coupled directly to the three-way stopcock.

In some embodiments, the erythrocyte-depleted plasma or serum traverses to and through a filter assembly that captures remaining aggregates but permits serum or plasma to flow through, into the collection vessel. In some embodiments, before the plasma or serum flows into the collection vessel, it flows through a surface filter comprising pores which are axially aligned with the collection vessel. In some embodiments, the plasma or serum flows through a depth filter comprising pores which are perpendicularly aligned to the flow of fluid into the collection vessel. In some embodiments, the filter assembly captures residual erythrocyte aggregates in the erythrocyte-depleted plasma and permits passage of plasma components such as platelets. Examples of filter materials include, but are not limited to, glass microfibers, a micro-porous membrane, and combinations thereof.

In step 150, in some embodiments, the collection vessel may be pre-filled with a preservative solution which is intended to reduce bioburden or to prevent and retard the growth and limit the survival and pathogenicity of microbial pathogens. In some embodiments, the preservative is coupled to the interior wall of the collection vessel. In some embodiments the preservative is coupled to a nanoparticle which can subsequently be removed during an in-line filtration step. In some embodiments, the preservative is a silver nanoparticle. In some embodiments, the preservative is added to the collection vessel after the serum or plasma has been collected.

In some embodiments, a platelet containing plasma is produced in the sedimentation vessel and collected in the collection vessel. In a variety of embodiments, the plasma produced contains an elevated level of platelets. In some embodiments, the concentration of platelets in the plasma is about 1.1 times greater than that of whole blood. In some embodiments, the concentration of platelets is at least about 1.1, at least about 1.2, at least about 1.3, at least about 1.4, at least about 1.5, at least about 1.6, at least about 1.7, at least about 1.8, at least about 1.9, or at least about 2 times greater than that of whole blood. In some embodiments, the platelet concentration in the plasma is at least about 2 times greater than the platelet concentration of the whole blood that was used to prepare the plasma. In some embodiments, the platelet concentration in the plasma is at least about 2 to 3 times greater than the platelet concentration of the whole blood that was used to prepare the plasma. In some embodiments, the platelet concentration in the plasma is at least about 3 to 10 times greater than the platelet concentration of the whole blood that was used to prepare the plasma. In some embodiments, the platelet concentration of the plasma is at least 10 times greater than that of centrifuged whole blood. In some embodiments, the plasma is at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, or at least about 40 times greater than that of centrifuged whole blood. In some embodiments, the platelet concentration of the plasma is at least about 3×10⁵, at least about 3.1×10⁵, at least about 3.2×10⁵, at least about 3.3×10⁵, at least about 3.4×10⁵, at least about 3.5×10⁵, at least about 3.6×10⁵, at least about 3.7×10⁵, at least about 3.8×10⁵, at least about 3.9×10⁵, at least about 4×10⁵, at least about 4.5×10⁵, at least about 5×10⁵, at least about 6×10⁵, at least about 7×10⁵, at least about 8×10⁵, at least about 9×10⁵, at least about 1×10⁶, platelets/μL

In some embodiments, the device is used to create a solution that is high in growth factors produced by platelets. In this case, the collection vessel may be pre-filled with a platelet-activating solution such as calcium chloride, adenosine diphosphate (ADP), thrombin, thromboxane A2, von Willebrand factor (vWF), collagen, chitosan, or arachidonic acid such that contact of the platelet-containing plasma with this platelet-activating solution results in release of growth factors from platelets. In some embodiments, the resulting platelet-containing plasma solution is cooled or heated to induce the release of growth factors contained within the platelets. In some embodiments the plasma is cooled to between −20and 20° C. In some embodiments, the plasma is heated to between 20 and 50° C. In some embodiments, freeze-thaw cycles of platelet containing plasma are used to activate platelets, causing them to release growth factors. In some embodiments, one freeze-thaw cycle is carried out. In some embodiments, two or more freeze-thaw cycles are carried out. In some embodiments, electric field stimulation of a solution of platelets in plasma is used to cause platelets to release growth factors. In some embodiments, ultrasound energy is used to cause platelets to release growth factors. In some embodiments, shear forces, such as passing the platelet-rich solution through a sieve, is used to cause platelets to release growth factors.

In some embodiments, the sedimentation vessel, sedimentation-accelerating solution, anticoagulant solution, preservative solution, filter(s), eye dropper bottle(s), one-way valve(s) and three-way stopcock are part of a kit.

In some embodiments, the kit's sedimentation vessel is a blood separation device in the form of a cylindrical tubular assembly similar in shape to a 6 ml Vacutainer™. In some embodiments, the kit's sedimentation vessel is a blood separation device in the form of a syringe.

In some embodiments, the kit's eye dropper bottle style collection vessel can be fitted with an eye dropper cap so as to facilitate maintenance of sterility and to allow for the bottle of product to be provided to the patient. In some embodiments, the eye dropper bottle style collection vessel comprises a pumping device for metering the eye drop volume.

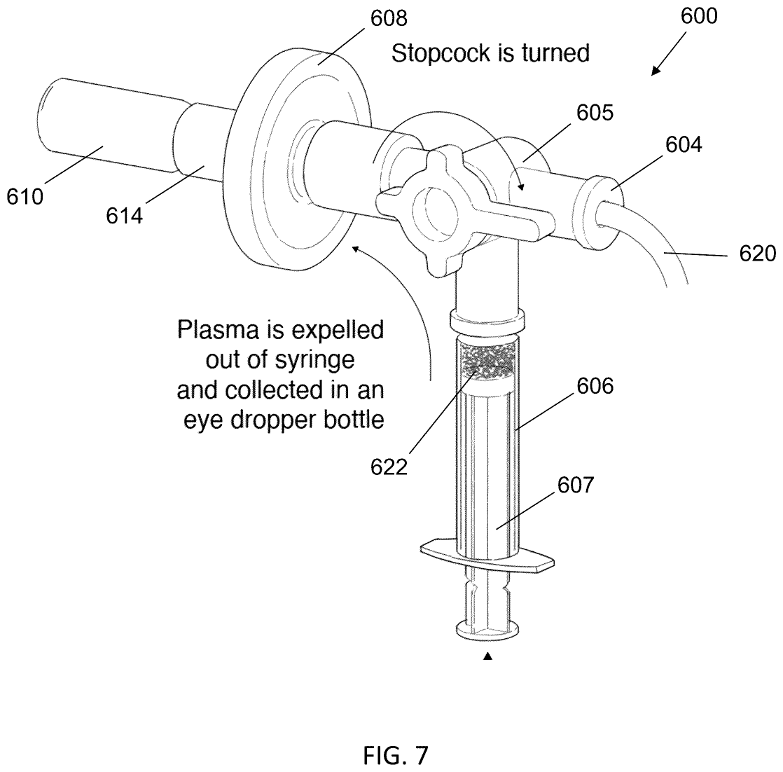

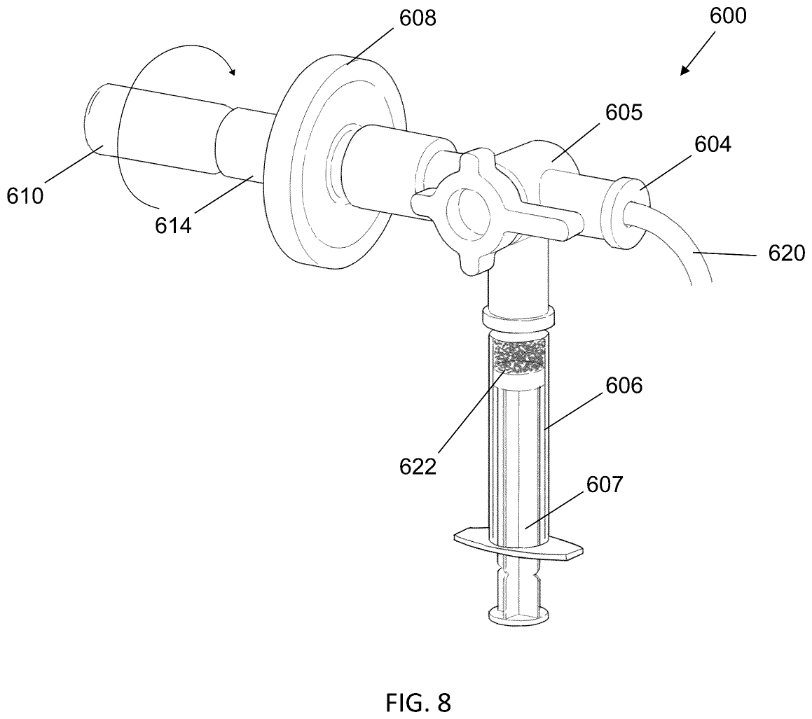

In some embodiments, the elements within a kit are assembled into a device (See for example FIG. 4). This device accomplishes the goal of producing serum or plasma eye drops using the following steps. Using standard venipuncture, the blood is drawn from a patient's vein through a one-way valve (See for example FIG. 3) into a channel 1 of a Luer lock three-way stopcock and then out of another channel 2 into a syringe-style hermetically sealed sedimentation vessel. As the blood is drawn into this sedimentation vessel, it mixes with a solution containing one or more sedimentation accelerating agent(s), an anticoagulant and a preservative which have been pre-filled in this sedimentation vessel (See for example FIG. 5). The stopcock is rotated to disconnect channel 1 from channel 2, the patient's vein and the one-way valve are detached from channel 1 of the three-way stopcock, and channel 1 of the three-way stopcock is sealed by any conventional means known in the art, such as a stopper, cap, or septum. The blood collection vessel and attached three-way stopcock, still hermetically sealed, is then placed on or in an inclined rack with the plunger side down. The mixture is then allowed to sediment for thirty minutes to one hour (See for example FIG. 6). Once the erythrocytes have sufficiently sedimented toward the plunger, supernatant (i.e., plasma or serum) is visible and collectable. In the next step, the knob of the three-way stopcock is turned to connect channel 2 with channel 3, the plunger of the sedimentation vessel is depressed, and the supernatant is then expelled through the three-way stopcock, through a filter and into the collection vessel (See for example FIG. 7). The filter removes microbes, leukocytes, and residual erythrocytes. The filter is directly connected to an eye dropper bottle style collection vessel and the erythrocyte-depleted serum or plasma is collected in this eye dropper bottle. If required, accumulated air pressure in the eye dropper bottle is vented by turning the three-way stopcock knob to disconnect all channels, a one-way valve placed on channel 1, and the three-way stopcock knob turned to connect channels 1 and 3. The collection vessel, once collection of the serum/plasma is completed, may be removed from the device by simple unscrewing of the vessel (See for example FIG. 8).

In some embodiments, the elements within a kit are assembled into a device that is a permutation of FIG. 4. This permuted device accomplishes the same goal as above, of producing serum or plasma eye drops using the following steps. Using standard venipuncture, the blood is drawn from a patient's vein through a one-way valve (See for example FIG. 3) into a channel 2 of a Luer lock three-way stopcock and then out of channel 1 into a syringe-style hermetically sealed sedimentation vessel. As the blood is drawn into this sedimentation vessel, it mixes with a solution containing one or more sedimentation accelerating agent(s), an anticoagulant and a preservative which have been pre-filled in this sedimentation vessel. In some embodiments, the patient's vein and the one-way valve are detached from channel 2 of the three-way stopcock, and channel 2 of the three-way stopcock is immediately sealed by any conventional means known in the art, such as a stopper, cap, or septum. The blood sedimentation vessel, the blood collection vessel and attached three-way stopcock, all still hermetically sealed, is then placed on or in an inclined rack with the syringe sedimentation vessel plunger side down. The mixture is then allowed to sediment for thirty minutes to one hour (See for example FIG. 6). Once the erythrocytes have sufficiently sedimented toward the plunger, supernatant (i.e., plasma or serum) is visible and collectable. In the next step, the knob of the three-way stopcock is turned 90 degrees to connect channel 1 with channel 3 (without an intervening step where channel 3 is connected to channel 2. In some embodiments, the collection vessel is pre-evacuated, causing plasma to flow into the collection vessel and in some embodiments the plunger of the sedimentation vessel is depressed, and the supernatant is then expelled through the three-way stopcock, possibly through flow limiting tubing, possibly through a filter into the collection vessel (See for example FIG. 7). In some embodiments, the filter removes microbes, leukocytes, and residual erythrocytes. The filter is directly connected to an eye dropper bottle style collection vessel and the erythrocyte-depleted serum or plasma is collected in this eye dropper bottle. In some embodiments, this eye dropper bottle is pre-filled with a platelet activating agent. In some embodiments a subsequent step such as electrical field stimulation, ultrasound If required, accumulated air pressure in the eye dropper bottle is vented by turning the three-way stopcock knob to disconnect all channels, a one-way valve placed on channel 1, and the three-way stopcock knob turned to connect channels 1 and 3. The collection vessel, once collection of the serum/plasma is completed, may be removed from the device by simple unscrewing of the vessel (See for example FIG. 8).

In some embodiments, step 150 further comprises the step of examining the erythrocyte-depleted plasma for the presence of pathogens such as viruses, fungi or bacteria. Contamination of the serum/plasma may have happened, for example, during the blood collection. In particular, the incorporation of pathogens is possible after inappropriate skin disinfection, and/or when there are even minor leaks in the hermetic seal of the sedimentation vessel or the collection vessel or any apparatus therewith.

In some embodiments, the examination of the serum/plasma represents additional safety in order to ensure improved product safety. In some embodiments the examination of the serum/plasma for pathogens and other contaminants represents a quality control step in the manufacturing of the apparatus. In some embodiments, the examination for contaminants is carried out after the storage. In other embodiments, examination for contaminants may be performed immediately before applying the erythrocyte-depleted plasma to the eye of the subject.

In some embodiments, step 150 further comprises the step of storing the erythrocyte-depleted serum or plasma. In some embodiments, the serum or plasma is stored at a temperature of 0° C. In some embodiments, the serum or plasma is stored at −20° C.

In some embodiments, the kit includes an apparatus to store the serum or plasma tears, maintaining it at a temperature below room temperature. In some embodiments, the serum or plasma is stored temporarily in a temperature-controlled refrigerator, such as until the results of the infection examination are available. In some embodiments, if the infection tests show negative results, the corresponding erythrocyte-depleted plasma can be applied safely to the eye of the subject.

In some embodiments, the application of the erythrocyte-depleted plasma to the eye of the subject in step 150 may be performed using any method known to those in the art.

### Device

Aspects of the present invention relate to a device for producing a topical serum or plasma ophthalmic solution. Referring now to FIG. 3, shown is an exploded view of an exemplary device 600. In some embodiments, device 600 comprises one or more three-way valve 605 comprising a first 601, second 602, and third 603 channel. Controlling fluid flow between channels of one or more three-way valve 605 is accomplished by turning the knob to connect the desired channels. Device 600 further comprises one or more one-way valve 604 comprising a first and second opening, wherein the second opening is fluidly and sealingly connected to a channel of three-way valve 605 (e.g., first channel 601), and configured to allow fluid to pass from the channel into three-way valve 605, but restrict flow in the opposite direction. Additionally, device 600 comprises a sedimentation vessel 606 having a cavity with an opening, wherein the opening is fluidly and sealingly connected to a channel (e.g., second channel 602) of three-way valve 605. In some embodiments, sedimentation vessel 606 further comprises a plunger 607 disposed within the cavity of sedimentation vessel 606, configured to increase and decrease the volume of the cavity per the actuation of the plunger. Further, device 600 comprises a filter 608 comprising a first and second opening, wherein the first opening is fluidly and sealingly connected to a channel (e.g., third channel 603) of three-way valve 605. Device 600 further comprises a receptacle 610 having a cavity and an opening, the opening of said receptacle fluidly and sealingly connected to the second opening of filter 608. In some embodiments, device 600 further comprises a tubular coupler 614 having a first and second opening, wherein the coupler is placed between filter 608 and receptacle 610, the first opening of the coupler fluidly and sealingly connected to the second opening of filter 608, and the second opening of the coupler fluidly and sealingly connected to the opening of receptacle 610. In some embodiments, tubular coupler comprises one or more flow rate reducing features disposed within the hollow interior region of the coupler. In some embodiments, an additional valve is sealingly and fluidly connected between three-way valve 606 and tubular coupler 614, or between tubular coupler 614 and receptacle 610.

The components of device 600 may be connected, sealed and/or fixed in place via a removable attachment that holds components in their respective positions, and fluidly connects components to other components, using any interfaces and/or methods known in the art that may create a water-tight and air-tight seal. This includes, but is not limited to, compression fits, friction fits, threads, collars, cams, locks, Luer locks, and the like. For example, in some embodiments, coupler 614 may comprise a gasket 616 positioned at the first opening of coupler 614, to fix and seal the attachment of coupler 614 to filter 608 with a compression fit. In another example, coupler 614 comprises threads 618 positioned near the second opening of coupler 614, wherein threads 612 of receptacle 610 may rotatably engage with threads 612 positioned near the opening of receptacle 610, to form a sealed fluid connection.

Referring now to FIG. 4, device 600 may be configured to operate in configurations as determined by the user-set positions of three-way valve 605. Three-way valve 605 comprises 3 connective positions that may be rotatably set using a switch, wherein the first 601, second 602, and third 603 channels are fluidly connected and/or disconnected depending on the setting. In some embodiments, the switch is a rotatably set stopcock valve as would be known by one of ordinary level of skill in the art. When three-way valve 605 is set to a first position, the first channel 601 is in fluid connection with the second channel 602, and both channels are fluidly disconnected from the third channel. In a second position, the second channel 602 is fluidly connected to the third channel 603, and both channels are fluidly disconnected from flow from the first channel 601, with the addition of a stopper or plug inhibiting flow through the first channel. In a third position, the first 601 and third 603 channels are fluidly connected, and fluidly disconnected from the second channel 602. When setting the switch from the first position to the second position, to prevent flow via the first channel, the switch is first rotated so that all three channels 601, 602, and 603 are blocked, and first channel 601 is then sealed. This may be accomplished with any manner known in the art, including placement of a stopper, cap, or septum on first channel 601.

In some embodiments, device 600 comprises one or more electronic valves, wherein the valve comprises at least one of an actuator, servo, motor, and the like. In some embodiments, the one or more electronic valves is connected to a controller, wherein commands from the controller adjust the position of the electronic valve and fluidly connect or disconnect at least a first and second channel of the valve. In some embodiments, three-way valve 605 is an electronically controlled valve. In some embodiments, device 600 comprises one or more pumps, wherein the pumps may be used to apply a vacuum and/or pressure to at least a portion of device 600. In some embodiments, the one or more pumps are connected to the controller also connected to the one or more electronic valves. In some embodiments, device 600 further comprises one or more one sensor. In some embodiments, the one or more sensor is at least one of a pressure sensor, a temperature sensor, a weight sensor, a light sensor, a flow sensor, and any sensor as would be known by one of ordinary level of skill in the art.

Again referring to FIG. 4, device 600 may fluidly connect to an inlet tubing 620, wherein inlet tubing fluidly connects to the first opening of one-way valve 604. In some embodiments, inlet tubing provides at least one fluid to device 600. In some embodiments, the fluid is blood. In some embodiments, reservoir 606 comprises at least one agent 622 contained within the cavity of the reservoir. In some embodiments, agent 622 comprises a sedimentation-accelerating solution containing one or more sedimentation-accelerating agent(s). In some embodiments, agent 622 additionally comprises an anticoagulant and/or preservative. In some embodiments, sedimentation vessel 606 is a medical syringe comprising a plunger 607. In some embodiments, receptacle 610 is an eye drop bottle. In some embodiments, sedimentation vessel 606 is a blood bag.

### Methods of Lubricating the Eye of a Subject

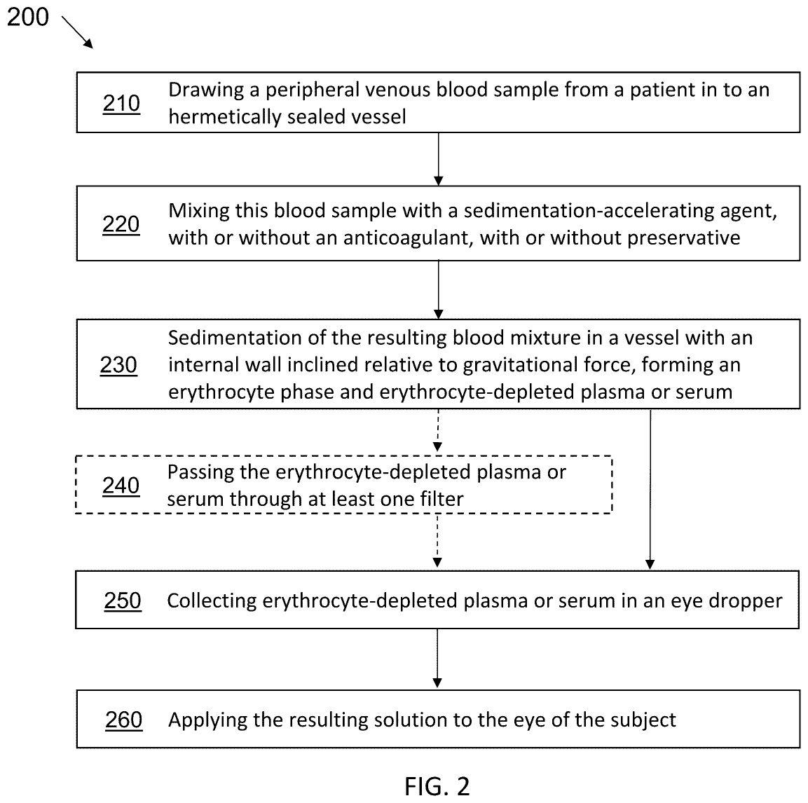

In another aspect, the present invention relates to a method of lubricating the eye of a subject using the collected, filtered plasma/serum solution. Exemplary method 200 is provided in FIG. 2. In step 210, a blood sample is drawn from the subject. In step 220, a sedimentation-accelerating agent and an anticoagulant are mixed with the blood sample to phase separate blood into an erythrocyte phase and an erythrocyte-depleted plasma or serum. In optional step 230, the erythrocyte-depleted plasma is passed through at least one filter. In step 240, the serum or plasma ophthalmic solution is collected directly into an eye dropper bottle. In step 250, the filtered erythrocyte-depleted plasma or serum is applied to the eye of the subject.

In some embodiments, the application of the erythrocyte-depleted plasma to the eye of the subject in step 250 may be performed using any method known to those in the art.

### Serum and Plasma of the Invention

In another aspect, the present invention relates to filtered serum and/or plasma produced using the methods described herein.

The erythrocyte-depleted serum or plasma prepared by the present method can contain less than 200,000 RBCs per microliter. For example, in various embodiments, the erythrocyte-depleted plasma prepared by the present methods may be 10-60% of the volume of the whole blood from which it is prepared, and contains less than 250,000, less than 200,000, less than 150,000, less than 125,000, less than 100,000, less than 75,000, or less than 50,000, or less than 10,000, or less than 1000, or less than 100, or less than 10, or less than 2 erythrocytes per microliter of erythrocyte-depleted plasma.

The erythrocyte-depleted plasma prepared by the present method can be enriched for platelets, such that the concentration of platelets in the collected plasma is greater than that of the whole blood from which it was prepared. For example, in various embodiments, the erythrocyte-depleted plasma prepared by the present methods may harbor 2 times, 3 times, 4 times, 5 times, 6 times, 7 times, 8 times, 9 times, or 10 times the platelet concentration of the whole blood from which it is prepared.

In some embodiments, the erythrocyte-depleted plasma further comprises some quantity of the sedimentation-accelerating agent. In some embodiments, the sedimentation-accelerating agent is known in the art to be safe for application to the eye surface. In some embodiments, the serum or plasma solution to be applied to the eye surface contains hemoglobin.

It should be appreciated that, while the serum or plasma described herein is in the context of eye drops, the serum or plasma prepared by the disclosed methods can be used for any purpose, or by any means, known in the art. As an example, in some embodiments, the serum or plasma prepared as described herein is used for analyses known in the art. Examples of such analysis include, but are not limited to, measurement of electrolyte levels, glucose levels, protein levels, or biomarker levels.

In some embodiments the platelet rich plasma produced by the methods described herein is applied topically to the skin. In some embodiments the platelet rich plasma produced by the methods described herein is used for dermatologic treatments. In some embodiments, the dermatologic treatments are used to treat the dermis.

In some embodiments, the platelet rich plasma produced by the methods described herein, or other derivatives of said platelet rich plasma, is used for joint injections (e.g., hip, knee, or shoulder). Examples of situations where said joint injections may be useful include, but are not limited to, sports medicine, pain management, rheumatology, and orthopedic surgery.

In some embodiments, the platelet rich plasma produced by the methods described herein is processed and used as a hemostatic agent. In some embodiments, the hemostatic agent is platelet-rich fibrin. In some embodiments, the resulting platelet-rich fibrin is used as a topical agent in wound care. In some embodiments, platelet-rich plasma or derivative preparations are used during surgery as a hemostatic or as a tissue glue.

### Kits of the Invention

In another aspect, the present invention relates to a kit for producing plasma or serum from the blood of a subject. In some embodiments, the plasma or serum is suitable for use as eyedrops. In some embodiments, the kit comprises a sedimentation vessel, a sedimentation-accelerating agent solution, an anticoagulant solution, a preservative solution, a platelet-activating solution, one or more three-way stopcocks, one or more one-way valves, a venipuncture set, a syringe or multiple syringes, a filtration apparatus such as a syringe filter, a plasma collection container complete with a nozzle, a coupler to connect the three-way stopcock to the container or to the filter, and a dispensing cap. In some embodiments, the container comprises a removable eye dropper bottle element. In some embodiments, the kit further comprises instructions which direct the use of the materials contained therein. In some embodiments, the kit includes an apparatus to keep the product of the invention, serum or plasma, in a temperature-controlled environment that is below room temperature.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

As centrifugation is among the rate limiting steps in the production of serum or plasma tears, technology which circumvents this step could, in theory, lead to faster preparation of serum or plasma eye drops. Similarly, as centrifugation requires expertise in laboratory techniques, technology which circumvents this step could, in theory, lead to less expensive preparation of serum or plasma eye drops, as specially trained technicians are not required for preparation. Centrifugation is a step in the preparation of serum or plasma eye drops that is carried out in order to remove erythrocytes (red blood cells) from blood products. Centrifugation may be thought of as increasing the force of gravity, and thus, according to Stokes' law, causes denser components of a homogenous solution (such as erythrocytes and leukocytes in blood) to accumulate in the portion of the centrifugation vessel that is furthest from the axis of rotation of the centrifuge.

Although centrifugation is the most widely used laboratory method, it is not the only method that can be used to separate erythrocytes from other blood components. When anticoagulated blood is maintained under low shear stress conditions (e.g., when blood is allowed to stand undisturbed in a container), erythrocytes will form aggregates. In some cases, these aggregates may be the result of agglutination by antibodies in the blood. In some cases, these aggregates are due to the electrostatic charge on the surface of erythrocytes causing cell-cell adhesion. Aggregates of this sort may look microscopically like a stack of coins and are thus referred to as rouleaux. As rouleaux and other aggregates are of higher density than surrounding solution, these aggregates will, over time, descend under the force of gravity, and will separate from the surrounding plasma. Clinical measurement of the extent and rate of formation of erythrocyte aggregates is referred to as measurement of the “erythrocyte sedimentation rate” or ESR.

In humans, there are known factors secreted in the body that, when present in the blood in higher-than-normal concentrations, will result in accelerated formation of rouleaux and other aggregates. One such factor is fibrinogen. Other such factors are immunoglobulins such as IgG and IgM. Other “acute phase reactants,” substances known to be indicative of a systemic inflammatory condition, such as rheumatoid arthritis or temporal arteritis, have been associated with increased rate and extent of rouleaux and other aggregate formation, and thus have been associated with increased ESR.

Addition of these substances exogenously to anticoagulated blood will cause an increase in the ESR. There are many substances known that will increase the formation of rouleaux and other aggregates, and will thus increase ESR.

The invention described herein leverages the use of substances that increase erythrocyte sedimentation via, among other mechanisms, aggregate formation. These substances, when added to anticoagulated whole blood in sufficient quantities, will obviate the need for centrifugation to separate erythrocytes from the surrounding plasma or serum solution.

### Example 1: Rapid and Cost-Effective Production of Serum or Plasma Eye Drops

The invention described herein will decrease the cost and time to acquisition of serum or plasma tears by eliminating the need for centrifugation and laboratory preparation. The process begins with blood drawn by peripheral venipuncture, collected through one channel of a three-way stopcock and allowed to flow through a second channel into a 50 mL Luer lock syringe pre-filled with approximately 5 mL of anticoagulant mixed with a sedimentation accelerating agent. 45 mL of blood is collected. The sedimentation accelerating solution may (in part) do so by increasing erythrocyte agglutination and/or formation of aggregates such as rouleaux, while the anticoagulant prevents clotting of the sample and the preservative prevents microbial growth.

Of note, the sedimentation accelerating solution may contain a mixture of both polymer and non-polymer solutions. Regarding the polymer solution, this may itself be a mixture of polymers of varying molecular weights. Dextran is a common and safe polymer ingredient which has been used to lubricate the eye surface in commercial preparations of artificial tear eye drops. Dextran happens also to be a highly potent accelerator of erythrocyte sedimentation, a phenomenon that is thought to take place in part because dextran accelerates induces rouleaux formation. Similarly, other ophthalmic safe polymers that may accelerate erythrocyte sedimentation include hydroxypropyl methylcellulose, polyethylene glycol and polyvinyl pyrrolidone (PVP, povidone).

In addition to a polymer, the aggregating solution may also contain a protein accelerator of sedimentation such as fibrinogen, an agglutinating agent of erythrocytes such as IgG or IgM, or an enzyme activator of erythrocyte sedimentation, such as bromelain or neuraminidase. The sedimentation-accelerating solution also may contain non-polymer, non-protein erythrocyte sedimentation accelerators such as the bioactive lipid lysophosphatidic acid.

Possible anticoagulants mixed with the sedimentation solution include heparin, sodium citrate or EDTA.

Possible preservatives mixed with the sedimentation accelerating solution include benzalkonium or sodium chlorite.

Once the blood has been collected in the syringe sedimentation vessel, it is mixed with the sedimentation accelerating agent(s), the anticoagulant and the preservative solutions. The syringe is incubated on a rack that is oriented at an incline in relation to the force of gravity such that the interior walls of the syringe are oriented at an angle of approximately 60 degrees in relation to gravity, and such that the plunger side of the syringe is below the outlet. The apparatus is allowed to stand at room temperature for approximately 30 to 60 minutes, to allow the serum/plasma phase to separate from the erythrocyte phase. The erythrocyte phase will collect near the plunger and the serum/plasma solution will be enriched near the syringe outlet.

Next, keeping the syringe in this position, the blood inflow channel of three-way stopcock is sealed, and the stopcock knob is turned to connect the syringe channel to flow limiting tubing and a 5-micron sieve filter which is, in turn, coupled to a pre-evacuated collection vessel that doubles as an eye dropper bottle.

The pre-evacuated eye dropper bottle (collection vessel), once the three-way stopcock's channel is connected to the syringe (sedimentation vessel), results in the generation of a pressure gradient which drives flow of erythrocyte-depleted serum/plasma solution into the 5-micron filter. The purpose of flow limiting tubing is to maintain low shear stress as the serum/blood flows through the device, thereby minimizing disruption of any residual RBC aggregates in the serum/plasma solution. The 5-micron filter acts as a sieve, limiting passage of any remaining erythrocyte aggregates, further depleting erythrocytes and further enriching the flowing solution with serum/plasma.

The plasma/serum solution flows out from the 5-micron filter into a coupler that connects it to an eye dropper bottle (collection vessel). Once collection is complete, the eye dropper bottle can be uncoupled, capped and dispensed to the patient.

In sum, all components are provided as a kit. Whole blood is drawn from a patient into a syringe pre-filled with a preservative, an anticoagulant, polymer sedimentation accelerator(s) (ex. dextran, PEG, or PVP) and potentially one or more non-polymer sedimentation accelerator(s). The syringe may be inverted gently several times to mix the blood with the solution and then this mixture is positioned at an incline in relation to the force of gravity and allowed to phase separate. After some period of time, typically between 30 and 60 minutes, the solution will have phase separated sufficiently, leaving erythrocytes descended to near the syringe plunger and serum/plasma in the supernatant near the syringe outlet. The resultant erythrocyte depleted serum/plasma is then passed out of the syringe, leaving the aggregated erythrocytes in the syringe. The serum/plasma is passed possibly into flow limiting tubing, possibly through a 5-micron or other filter to remove any residual erythrocytes and leukocytes. The serum/plasma solution is collected in a pre-evacuated eye dropper bottle via a coupler that connects the filter to the eye dropper bottle. In the case that plasma is collected, the eye dropper may be pre-filled with a platelet-activating solution to induce release of growth factors. This solution may then be applied to the eye of a subject using the eye dropper bottle.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A method of producing plasma solution, the method comprising the steps of:
drawing a blood sample from a subject;
adding one or more sedimentation-accelerating agent(s) and an anticoagulant to the blood sample to form an erythrocyte aggregate and an erythrocyte-depleted plasma; and
collecting the erythrocyte-depleted plasma.

2. The method of claim 1, wherein the blood sample, sedimentation-accelerating agent(s), and erythrocyte-depleted plasma are not exposed to the ambient atmosphere.

3. The method of claim 2, wherein the erythrocyte-depleted plasma is brought to ambient pressure by introducing the plasma to pathogen-free air.

4. The method of claim 1, wherein the blood sample is not exposed to centrifugation.

5. The method of claim 1, further comprising the step of passing the erythrocyte-depleted serum or plasma through at least one filter.

6. The method of claim 1, wherein the sedimentation-accelerating agent comprises a nanoparticle, polymer, macromolecule, protein, proteolytic enzyme, a small molecule or a combination thereof.

7. The method of claim 1, wherein the sedimentation-accelerating agent comprises a polymer conjugated to a nanoparticle.

8. The method of claim 1, wherein the sedimentation-accelerating agent comprises dextran having a molecular weight ranging from 40 kDa to 1000 kDa.

9. The method of claim 1, wherein the sedimentation-accelerating agent comprises at least two compounds selected from the group consisting of a nanoparticle, polymer, a protein, an enzyme, a proteolytic enzyme and a small molecule.

10. The method of claim 1, wherein the sedimentation-accelerating agent is immobilized on a solid-phase immobilization material.

11. A method of lubricating the eye of a subject, the method comprising the steps of:
drawing a blood sample from a subject;
adding a sedimentation-accelerating agent and an anticoagulant to the blood sample to form an erythrocyte aggregate and an erythrocyte-depleted plasma;
collecting the erythrocyte-depleted plasma; and
applying the erythrocyte-depleted plasma to the eye of the subject.

12. The method of claim 11, wherein the step of collecting the erythrocyte-depleted plasma further comprises the step of mixing the plasma with a platelet-activating agent.

13. The method of claim 11, wherein the sedimentation-accelerating agent comprises a nanoparticle, polymer, macromolecule, protein, proteolytic enzyme, a small molecule, or a combination thereof.

14. The method of claim 11, further comprising the step of treating the resulting platelet-rich plasma with a method selected from the group consisting of: ultrasound, an electric field, heating, cooling, heating and cooling, shear forces and treating with a substance selected from the group consisting of: calcium chloride, adenosine diphosphate (ADP), thrombin, thromboxane A2, von Willebrand factor (vWF), collagen, chitosan, and arachidonic acid;
wherein said treatment method activates platelets to release growth factors.

15. A kit for producing plasma from a blood sample, the kit comprising: a container comprising an erythrocyte sedimentation accelerating solution; a three-way stop cock; a venipuncture set; a syringe; a filter; and a plasma collection container; a coupler connecting the container to the three-way stopcock or to the filter; a dispensing cap; and instructions for use of the kit.

16. The kit of claim 15, wherein the erythrocyte sedimentation accelerating solution comprises a nanoparticle, polymer, macromolecule, protein, proteolytic enzyme, a small molecule, or a combination thereof.

17. The method of claim 1, wherein the sedimentation-accelerating agent comprises a polymer conjugated to a nanoparticle.

18. The method of claim 1, further comprising the step of treating the resulting platelet-rich plasma with a method selected from the group consisting of: ultrasound, an electric field, heating, cooling, heating and cooling, shear forces and treating with a substance selected from the group consisting of: calcium chloride, adenosine diphosphate (ADP), thrombin, thromboxane A2, von Willebrand factor (vWF), collagen, chitosan, and arachidonic acid;
wherein said treatment method activates platelets to release growth factors.

19. The method of claim 1, wherein the plasma solution is ophthalmic plasma solution.

20. The kit of claim 15, wherein the plasma is suitable for use as eye drops and wherein the container is an eye dropper bottle element.
